(19) United States
(12) Patent Application Publication
(10) Pub. No.: US 2002/0155540 A1
(43) Pub. Date: Oct. 24, 2002

(54) Method to reduce transcriptional interference between tandem genes

(76) Inventor: Malla Padidam, Chalfont, PA (US)

Correspondence Address: ROHM AND HAAS COMPANY, PATENT DEPARTMENT, 100 INDEPENDENCE MALL WEST, PHILADELPHIA, PA, 19106-2399, US

(21) Appl. No.: 10/074,744

(22) Filed: Feb. 13, 2002

Related U.S. Application Data
(60) Provisional application No. 60/268,584, filed on Feb. 14, 2001.

(51) Int. Cl. C12P 21/02; C12N 15/87

(52) U.S. Cl. 435/69.1; 435/455; 435/320.1

(57) ABSTRACT

This invention relates to the field of biotechnology or genetic engineering. Specifically, this invention relates to the field of gene expression. More specifically, this invention relates to methods to reduce or eliminate transcriptional interference between two or more tandemly arranged genes within a host cell.

This application claims priority to co-pending U.S. provisional application serial number 60/268,584, filed Feb. 14, 2001.

### FIELD OF THE INVENTION

This invention relates to the field of biotechnology or genetic engineering. Specifically, this invention relates to the field of gene expression. More specifically, this invention relates to methods to reduce or eliminate transcriptional interference between two or more tandemly arranged genes within a host cell.

### BACKGROUND OF THE INVENTION

Plant biologists routinely express transgenes in plant cells. Very often, two or more genes are cloned next to each other as tandemly arranged genes on a plasmid before the plasmid is introduced into plant cells. The genes of interest are cloned between the promoter and terminator/polyadenylation [poly(A)] sequences to create gene expression cassettes. The orientation of the cassettes is usually determined by the availability of convenient restriction enzyme sites on the plasmid.

Transcriptional interference refers to the phenomenon of reduced expression of a gene caused by the transcription from an upstream or downstream promoter and has been well documented in mammalian, yeast and prokaryotic systems (5, 6, 13). It can occur when different genes or gene expression cassettes are arranged in tandem, or even when these genes or cassettes are separated by several kilobases (5). The terminators in these gene constructs are not sufficient to prevent transcriptional interference between two adjacent genes. Possible mechanisms for reduction of transgene expression by an adjacent promoter include antisense RNA production that can co-suppress the endogenous copy of the transgene (7, 10), RNA polymerase “collision” (11), localized depletion of diffusible transcription factors, and DNA topological constraints (16).

Earlier studies in transgenic plants have shown that placing a promoter at the 3′ end of a gene expression cassette and in opposite orientation reduces the expression of the upstream gene (8, 11). A poly(A) signal sequence placed on either side of the transgene blocked the downstream transcriptional read-through and restored the upstream gene expression (8). Configuration of transfer DNA (T-DNA) and whether the genes are placed at the left or right border of the T-DNA of the binary plant transformation vector (position effect) were also shown to have effects on transgene expression in plants (2, 3, 12). Tetracycline-dependent activation of an upstream promoter was shown to reduce the downstream gene expression in tomato (16).

Even though the phenomenon of transcriptional interference is well known, the mechanism is poorly understood. Therefore, a need exists to provide methods to reduce transcriptional interference between tandemly arranged genes or expression cassettes to allow efficient or improved expression of these adjacent genes or cassettes. Applicant has now developed novel methods to overcome this problem and show herein that their novel methods effectively reduce or eliminate transcriptional interference between tandem genes. Various publications are cited herein, the disclosures of which are incorporated by reference in their entireties. The citation of any reference herein should not be construed as an admission that such reference is available as “Prior Art” to the instant application.

### SUMMARY OF THE INVENTION

The present invention relates to methods to reduce or eliminate transcriptional interference between two or more tandemly arranged genes or gene expression cassettes in a host cell.

Specifically, the present invention relates to a method to reduce or eliminate transcriptional interference between two or tandemly arranged genes or gene expression cassettes in a host cell comprising introducing into the host cell a polynucleotide comprising a first gene expression cassette encoding a first polypeptide, whereby the first gene expression cassette and the second gene expression cassette are positioned in a tandem orientation; and culturing the host cell under conditions, whereby transcriptional interference between the first gene expression cassette and the second gene expression cassette is reduced or eliminated and the first polypeptide and the second polypeptide are expressed. In a specific embodiment, the first gene expression cassette and the second gene expression cassette are positioned in a head (5′)-to-head (5′) orientation relative to each other.

The present invention also relates to a method to reduce or eliminate transcriptional interference between two tandemly arranged genes or gene expression cassettes in a host cell comprising introducing into the host cell a polynucleotide comprising a) a first gene expression cassette encoding a first polypeptide, b) a spacer polynucleotide, and c) a second gene expression cassette encoding a second polypetide, whereby the first gene expression cassette and the second gene expression cassette are positioned in a tandem orientation and the spacer polynucleotide of b) is positioned between the first expression cassette and the second expression cassette; and culturing the host cell under conditions, whereby transcriptional interference between the first gene expression cassette and the second gene expression cassette is reduced or eliminated and the first polypeptide and the second polypeptide are expressed.

In a specific embodiment, the first gene expression cassette and the second gene expression cassette are positioned in a tail (3′)-to-tail (3′) orientation relative to each other. In another specific embodiment, the first gene expression cassette and the second gene expression cassette are positioned in a head (5′)-to-tail (3′) orientation relative to each other.

In a preferred embodiment, the spacer polynucleotide of b) comprises a nucleic acid sequence selected from the group consisting of SEQ ID NO: 1[154bp Transcription Blocker (TB) sequence], SEQ ID NO: 2 (702 bp BSTEII lambda fragment), SEQ ID NO: 3 (1519 bp StuI lambda fragment), and SEQ ID NO: 4 (2322 bp HindIII lambda fragment).

In another preferred embodiment, the spacer polynucleotide of b) comprises at least a 40% adenine and thiamine nucleotide content. Preferably, the spacer polynucleotide of b) comprises and adenine and thiamine nucleotide content of at least 46%, 48%, or 63%. More preferably, the spacer polynucleotide of b) comprises a polynucleotide sequence selected from the group consisting of SEQ ID NO: 2 (702 by BstEII lambda fragment), SEQ ID NO: 3 (1519 by StuI lambda fragment), and SEQ ID NO: 4 (2322 bp HindIII lambda fragment).

The present invention also relates to a transformed host cell produced as a result of a method of the invention.

### BRIEF DESCRIPTION OF THE DRAWINGS

FIG. 1 Schematic representation of gene constructs. (A) Luciferase (LUC), green fluorescent protein (GFP) and the Renilla luciferase (FLUC) genes for plant expression are shown. Coding sequences for LUC, GFP, and RLUC are flanked on the 5′ end by the cassava vein mosaic virus promoter (CsVMV), the cauliflower mosaic virus promoter (35S), or the cauliflower mosaic virus duplicated promoter (E35S) and on the 3′ end by the 35S terminator (35St) or NOS terminator (NOSt). GFP gene in addition contained tobacco etch virus (TEV) enhancer sequence. All genes were cloned in pBluescriptII SK. (B) Plasmid constructs containing GFP and LUC genes head to tail (→→), head to head (←→), and tail-to-tail (→←). (C) Transcription blocker sequence (TB) or a λ DNA fragment is placed between the GFP and LUC genes. (D) Plasmid used for chemical induction of LUC gene. GVE is a chimeric protein consisting of GAL4 DNA binding, VP16 activation, and ecdysone receptor ligand binding domains. LUC coding sequence is placed behind the response element (where GVE can bind) consisting of 5 copies of GAL4 binding site (5×GAL) and miminal 35S promoter (M35S).

FIG. 2. Reduced expression of LUC gene due to the adjacent GFP gene. Protoplast were transfected with the constructs shown on the left along with the RLUC plasmid as an internal control. LUC and Rluc enzyme activities were measured in relative light units as described in Example 1.3. LUC expression levels are shown as ratios of LUC/RLUC activities. The error bars represent standard deviations based on 5 to 7 independent transfections.

FIG. 3. Activation of an upstream promoter using a chemically inducible system reduced the expression of the constitutive downstream gene. Protoplasts transfected with the plasmid shown in FIG. 1D were divided into two parts, and one part received no chemical (Uninduced and other part received 10 μM methoxyfenozide (Induced). LUC and RLUC activities are shown in relative light units (RLU). The error bars represent standard deviations based on 3 independent transfections.

FIG. 4. Transcriptional interference is eliminated by cloning transcription blocker or λ DNA sequence between the genes. The transcription blocker sequence (TB), a 702, 1,519, or 2322 by λ DNA fragments were cloned between GFP and LUC gene constructs shown on the left. Plasmid constructs were introduced into protoplasts along with the RLUC plasmid (as an internal control and LUC and RLUC activities were assayed. LUC activities are shown as ratio of LUC/RLUC activities. The error bars represent standard deviations based on 5 to 7 independent transfections.

FIG. 5. Generic configuration of ExpressIt™ inducible gene expression system.

FIG. 6. Inducible gene expression of GVE-Luc tail-to-tail construct in Arabidopsis T0 plants. Luc and RLuc activities were assayed. Luc activities are shown as ratio of Luc/RLuc activities.

FIG. 7. Inducible gene expression of GVE-Luc tail-to-tail construct in tobacco T0 plants. Luc and Rluc activities were assayed. Luc activities are shown as ratio of Luc/RLuc activities.

FIG. 8. Inducible gene expression of GVE-Luc head-to-tail construct in Arabidopsis T0 plants. Luc and Rluc activities were assayed. Luc activities are shown as ratio of Luc/RLuc activities.

FIG. 9. Inducible gene expression of GVE-Luc head-to-tail construct in Arabidopsis T1 plants. Luc and Rluc activities were assayed. Luc activities are shown as ratio of Luc/Rluc activities.

FIG. 10. Inducible gene expression of GVC-Luc head-to-tail construct in tobacco T0 plants. Luc and Rluc activities were assayed. Luc activities are shown as ratio of Luc/RLuc activities.

FIG. 11. Inducible gene expression of GVE-Luc head-to-head construct in Arabidopsis T0 plants. Luc and RLuc activities were assayed. Luc activities are shown as ratio of Luc/Rluc activities.

FIG. 12. Inducible gene expression of GVE-Luc head-to-head construct in Arabidopsis T1 plants. Luc and RLuc activities were assayed. Luc activities are shown as ratio of Luc/Rluc activities.

FIG. 13. Inducible gene expression of GVE-Luc head-to-head construct in tobacco T0 plants. Luc and Rluc activities were assayed. Luc activities are shown as ratio of Luc/RLuc activities.

### DETAILED DESCRIPTION OF THE INVENTION

Very often plants are transformed with two or more gene expression cassettes cloned in tandem. However, transcriptional interference between the two or more tandemly arranged cassettes can occur, resulting in decreased polypeptide production from one or more of the cassettes. Applicant has now shown that this transcriptional interference can be reduced or eliminated by modifying the orientation of the gene expression cassettes and/or by placing a spacer polynucleotide sequence between the cassettes. The present invention provides novel methods to reduce or eliminate transcriptional interference between adjacent genes and can be used to improve expression of multiple gene expression constructs in both prokaryotic and eukaryotic host cells. Thus, Applicant's novel methods overcome the limitations of tandemly arranged gene expression.

Definitions

In this disclosure, a number of terms and abbreviations are used. The following definitions are provided and should be helpful in understanding the scope and practice of the present invention.

In a specific embodiment, the term “about” or “approximately” means within 20%, preferably within 10%, more preferably within 5%, and even more preferably within 1% of a given value o range.

A “nucleic acid” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes but is not limited to cDNA, genomic DNA, plasmids DNA, synthetic DNA, and semi-synthetic DNA. DNA may be linear, circular, or supercoiled.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

As used herein, an “isolated nucleic acid fragment” is a polymer of RNA or DNA that is single-or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A “gene” refers to an assembly of nucleotides that encode a polypeptide, and includes cDNA and genomic DNA nucleic acids. “Gene” also refers to a nucleic acid fragment that expresses a specific protein, including regulatory sequences preceding (5′ non-coding sequences) and following (3′ non-coding sequences) the coding sequence. “Native gene” refers to any gene that is not a native gene, comprising regulatory and/or coding sequences that are not found together in nature. Accordingly, chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Heterologous” DNA refers to DNA not naturally located in the cell, or in a chromosomal site of the cell. Preferably, the heterologous DNA includes a gene foreign to the cell.

The term “genome” includes chromosomal as well as mitochondrial, chloroplast and viral DNA or RNA.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989 infra). Hybridization and washing conditions are well known and exemplified in Sambrook. J., Fristsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization.

Stringency conditions can be adjusted to screen for moderately similar fragments, such as homologous sequences from distantly related organisms, to highly similar fragments, such as genes that duplicate functional enzymes from closely related organisms. For preliminary screening for homologous nucleic acids, low stringency hybridization conditions, corresponding to a Tₘ of 55°, can be used, e.g., 5×SSC, 0.1% SDS, 0.25% milk, and no formamide; or 30% formamide, 5×SSC, 0.5% SWDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. Hybridization requires that the two nucleic acids contain complementary sequences, although depending on the stringency of the hybridization, mismatches between bases are possible.

The term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid fragments that are complementary to the complete sequences as disclosed or used herein as well as those substantially similar nucleic acid sequences.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

Post-hybridization washes also determine stringency conditions. One set of preferred conditions uses a series of washes starting with 6×SSC, 0.5% SDS at room temperature for 15 min, then repeated with 2×SSC. 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60°0 C. Another preferred set of highly stringent conditions uses two final washes in 0.1×SSC, 0.1% SDS at 65° C. Hybridization requires that the two nucleic acids comprise complementary sequences, although depending on the stringency of the hybridization, mismatches between bases are possible.

The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8).

In one embodiment the length for a hybridizable nucleic acid is at least about 10 nucleotides. Preferable a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

The term “probe” refers to a single-stranded nucleic acid molecule that can base pair with a complementary single stranded target nucleic acid to form a double-stranded molecule.

As used herein, the term “oligonucleotide” refers to a nucleic acid, generally of at least 18 nucleotides, that is hybridizable to a genomic DNA molecule, a cDNA molecule, a plasmid DNA or an mRNA molecule. Oligonucleotides can be labeled, e.g., with ³²P-nucleotides or nucleotides to which a label, such as biotin, has been covalently conjugated. A labeled oligonucleotide can be used as a probe to detect the presence of a nucleic acid. Oligonucleotides (one or both of which may be labeled) can be used as PCR primers, either for cloning full length or a fragment of a nucleic acid, or to detect the presence of a nucleic acid. An oligonucleotide can also be used to form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “primer” is an oligonucleotide that hybridizes to a target nucleic acid sequence to create a double stranded nucleic acid region that can serve as an initiation point for DNA synthesis under suitable conditions. Such primers may be used in a polymerase chain reaction.

“Polymerase chain reaction” is abbreviated PCR and means an in vitro method for enzymatically amplifying specific nucleic acid sequences. PCR involves a repetitive series of temperature cycles with each cycle comprising three stages: denaturation of the template nucleic acid to separate the strands of the target molecule, annealing a single stranded PCR oligonucleotide primer to the template nucleic acid, and extension of the annealed primer(s) by DNA polymerase. PCR provides a means to detect the presence of the target molecule and, under quantitative or semi-quantitative conditions, to determine the relative amount of that target molecule within the starting pool of nucleic acids.

“Reverse transcription-polymerase chain reaction” is abbreviated RT-PCR and means an in vitro method for enzymatically producing a target cDNA molecule or molecules from an RNA molecule or molecules, followed by enzymatic amplification of a specific nucleic acid sequence or sequences within the target cDNA molecule or molecules as described above. RT-PCR also provides a means to detect the presence of the target molecule and, under quantitative or semi-quantitative conditions, to determine the relative amount of that target molecule within the starting pool of nucleic acids.

A DNA “coding sequence” is a double-stranded DNA sequence which is transcribed and translated into a polypeptide in a cell in vitro or in vivo when placed under the control of appropriate regulatory sequences. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence.

“Open reading frame” is abbreviated ORF and means a length of nucleic acid sequence, either DNA, cDNA or RNA, that comprises a translation start signal or initiation codon, such as an ATG or AUG, and a termination codon and can be potentially translated into a polypeptide sequence.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters”. Promoters that cause a gene to be expressed in a specific cell type are commonly referred to as “cell-specific promoters” or “tissue-specific promoters”. Promoters that cause a gene to be expressed at a specific stage of development or cell differentiation are commonly referred to as “developmentally-specific promoters” or “cell differentiation-specific promoters”. Promoters that are induced and cause a gene to be expressed following exposure or treatment of the cell with an agent, biological molecule, chemical, ligand, light, or the like that induces the promoter are commonly referred to as “inducible promoters” or “regulatable promoters”. It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from a nucleic acid or polynucleotide. Expression may also refer to translation of mRNA into a polypeptide.

The terms “cassette”, “expression cassette” and “gene expression cassette” refer to a segment of DNA that can be inserted into a nucleic acid or polynucleotide at specific restriction sites or by homologous recombination. The segment of DNA comprises a polynucleotide that encodes a polypeptide of interest, and the cassette and restriction sites are designed to ensure insertion of the cassette in the proper reading frame for transcription and translation. “Transformation cassette” refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

The term “head-to-head” is used herein to describe the orientation of two nucleotide sequences, particularly two gene expression cassettes, in relation to each other. Two gene expression cassettes are positioned in a head-to-head orientation when the 5′ end of the coding strand of one gene expression cassette is adjacent to the 5′ end of the coding strand of the other gene expression cassette, whereby the direction of transcription of each gene expression cassette proceeds away from the 5′ end of the other expression cassette. The term “head-to-head” may be abbreviated (5′)-to-(5′) and may also be indicated by the symbols (←→) or (3′←5′5′→3′).

The term “tail-to-tail” is used herein to describe the orientation of two nucleotide sequences, particularly two gene expression cassettes, in relation to each other. Two gene expression cassettes are positioned in a tail-to-tail orientation when the 3′ end of the coding strand of one gene expression cassette is adjacent to the 3′ end of the coding strand of the other gene expression cassette, whereby the direction of transcription of each gene expression cassette proceeds toward the other expression cassette. The term “tail-to-tail” may be abbreviated (3′)-to-(3′) and may also be indicated by the symbols (→←) or (5′→3′3′←5′).

The term “head-to-tail” is used herein to describe the orientation of two nucleotide sequences, particularly two gene expression cassettes, in relation to each other. Two gene expression cassettes are positioned in a head-to-tail orientation when the 5′ end of the coding strand of one gene expression cassette is adjacent to the 3′ end of the coding strand of the other gene expression cassette, whereby the direction of transcription of each gene expression cassette proceeds in the same direction as that of the other expression cassette. The term “head-to-tail” may be abbreviated (5′)-to-(3′) and may also be indicated by the symbols (→→) or (5′→3′5′→3′).

The term “downstream” refers to a nucleotide sequence that is located 3′ to reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to reference nucleotide sequence. In particular, upstream nucleotide sequences generally relate to sequences that are located on the 5′ side of a coding sequence or starting point of transcription. For example, most promoters are located upstream of the start site of transcription.

The term “spacer polynucleotide” refers to a polynucleotide comprising a sequence of variable length and/or varying amounts of adenine and thiamine nucleotide content. The spacer polynucleotide may be inserted between two tandemly arranged gene expression cassettes according to the invention. The spacer polynucleotide may be heterologous DNA. In a specific embodiment, the spacer polynucleotide comprises at least 50 nucleotides or base pairs. Preferably, the spacer polynucleotide comprises at least 50, 75, 100, 125, 150, 250, 500, 750, 1000, 1250, 1500, 1750, 2000, 2250, 2500, 2750, or 3000 nucleotides or base pairs. More preferably, the spacer polynucleotide comprises 154, 702, 1519, or 2322 nucleotides or base pairs. In another specific embodiment, the spacer polynucleotide comprises at least 40% adenine and thiamine nucleotide content. Preferably, the spacer polynucleotide comprises at least 46% adenine and thiamine nucleotide content. More preferably, the spacer polynucleotide comprises at least 48% adenine and thiamine nucleotide content. Even more preferably, the spacer polynucleotide comprises at least 63% adenine and thiamine nucleotide content.

The terms “restriction endonuclease” and “restriction enzyme” refer to an enzyme that binds and cuts within a specific nucleotide sequence within double stranded DNA.

“Homologous recombination” refers to the insertion of a foreign DNA sequence into another DNA molecule, e.g., insertion of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. Viral vectors include retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A “cloning vector” is a “replicon”, which is a unit length of DNA that replicates sequentially and which comprises an origin of replication, such as a plasmid, phage or cosmid, to which another DNA segment may be attached so as to bring about the replication of the attached segment. Cloning vectors may be capable of replication in one cell type and expression in another (“shuttle vector”).

A cell has been “transfected” by exogenous or heterologous DNA when such DNA has been introduced inside the cell. A cell has been “transformed” by exogenous or heterologous DNA when the transfected DNA effects a phenotypic change. The transforming DNA can be integrated (covalently linked) into chromosomal DNA making up the genome of the cell.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “genetic region” will refer to a region of a nucleic acid molecule or a nucleotide sequence that comprises a gene encoding a polypeptide.

The term “selectable marker” means an identifying factor, usually an antibiotic or chemical resistance gene, that is able to be selected for based upon the marker gene's effect, i.e., resistance to an antibiotic, resistance to a herbicide, and the like, wherein the effect is used to track the inheritance of a nucleic acid of interest and/or to identify a cell or organism that has inherited the nucleic acid of interest. Examples of reporter genes known and used in the art include: genes providing resistance to streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like.

The term “reporter gene” means a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include: luciferase (Luc), green fluorescent protein (GFP), chloramphenicol acetyltransferase (CAT), β-galactosidase (LacZ), β-glucuronidase (Gus), and the like.

The plasmids or vectors according to the invention may further comprise at least one promoter suitable for driving expression of a gene in a host cell. The term “expression vector” means a vector, plasmid or vehicle designed to enable the expression of an inserted nucleic acid sequence following transformation into the host. The cloned gene, i.e., the inserted nucleic acid sequence, is usually placed under the control of control elements such as a promoter, a minimal promoter, an enhancer, or the like. Initiation control regions or promoters, which are useful to drive expression of a nucleic acid in the desired host cell are numerous and familiar to those skilled in the art. Virtually any promoter capable of driving these genes is suitable for the present invention including but not limited to: viral promoters, plant promoters, bacterial promoters, animal promoters, mammalian promoters, synthetic promoters, constitutive promoters, tissue specific promoter, developmental specific promoters, inducible promoters, light regulated promoters; CYC1, HIS3, GAL1, GAL4, GAL10, ADH1, PGK, PHO5, GAPDH, ADC1, TRP1, URA3, LEU2, ENO, TPI, alkaline phosphatase promoters (useful for expression in Saccharomyces); AOX1 promoter (useful for expression in Pichia); b-lactamase, lac, ara, tet, trp, lP_{L}, lP_{R}, T7, tac, and trc promoters (useful for expression in Escherichia coli); and light regulated-, seed specific-, pollen specific-, ovary specific-, pathogenesis or disease related-, cauliflower mosaic virus (CMV) 35S, CMV 35S minimal, cassava vein mosaic virus (CsVMV), chlorophyll a/b binding protein, ribulose 1, 5-bisphosphate carboxylase, shoot-specific, root specific, chitinase, stress inducible, rice tungro bacilliform virus, plant super-promoter, potato leucine aminopeptidase, nitrate reductase, mannopine synthase, nopaline synthase, ubiquitin, zein protein, and anthocyanin promoters (useful for expression in plant cells); animal and mammalian promoters known in the art include, but are not limited to, the SV40 early promoter region, the promoter contained in the 3′ long terminal repeat (LTR) of Rous sarcoma virus (RSV), the cytomegalovirus (CMV) early promoter, the herpes thymidine kinase (TK) promoter, the regulatory sequences of the metallothionein gene, and transcriptional control regions, which exhibit tissue specificity and have been utilized in transgenic animals, such as the elastase I gene control region which is active in pancreatic acinar cells; insulin gene control region which is active in pancreatic beta cells, immunoglobulin gene control region which is active in lymphoid cells, mouse mammary tumor virus control region which is active in testicular, breast, lymphoid and mast cells, albumin gene control region which is active in liver, alpha-fetoprotein gene control region which is active in liver, alpha 1-antitrypsin gene control region which is active in the liver, beta-globin gene control region which is active in myeloid cells, myelin basic protein gene control region which is active in oligodendrocyte cells in the brain, myosin light chain-2 gene control region which is active in skeletal muscle, and gonadotropic releasing hormone gene control region which is active in the hypothalamus, and the like. In a preferred embodiment of the invention, the promoter is selected from the group consisting of a cauliflower mosaic virus 35S promoter, a cassava vein mosaic virus promoter, and a cauliflower mosaic virus 35S minimal promoter.

Enhancers that may be used in embodiments of the invention include but are not limited to: tobacco mosaic virus enhancer, cauliflower mosaic virus 35S enhancer, tobacco etch virus enhancer, ribulose 1, 5-bisphosphate carboxylase enhancer, rice tungro bacilliform virus enhancer, and other plant and viral gene enhancers, and the like.

Termination control regions, i.e., terminator or polyadenylation sequences, may also be derived from various genes native to the preferred hosts. Optionally, a termination site may be unnecessary, however, it is most preferred if included. In a preferred embodiment of the invention, the termination control region may be comprise or be derived from a synthetic sequence, nopaline synthase (nos), cauliflower mosaic virus (CaMV), octopine synthase (ocs), Agrobacterium, viral, and plant terminator sequences, or the like.

The terms “3′ non-coding sequences” or “3′ untranslated region (UTR)” refer to DNA sequences located downstream (3′) of a coding sequence and may comprise polyadenylation [poly(A)] recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“Regulatory region” means a nucleic acid sequence which regulates the expression of a second nucleic acid sequence. A regulatory region may include sequences which are naturally responsible for expressing a particular nucleic acid (a homologous region) or may include sequences of a different origin which are responsible for expressing different proteins or even synthetic proteins (a heterologous region). In particular, the sequences can be sequences of prokaryotic, eukaryotic, or viral genes or derived sequences which stimulate or repress transcription of a gene in a specific or non-specific manner and in an inducible or non-inducible manner. Regulatory regions include origins of replication, RNA splice sites, promoters, enhancers, transcriptional termination sequences, and signal sequences which direct the polypeptide into the secretory pathways of the target cell.

A regulatory region from a “heterologous source” is a regulatory region which is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene. The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

A “polypeptide” is a polymeric compound comprised of covalently linked amino acid residues. Amino acids have the following general structure:

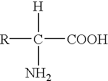

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. A polypeptide of the invention preferably comprises at least about 14 amino acids.

A “protein” is a polypeptide that performs a structural or functional role in a living cell.

A “variant” of a polypeptide or protein is any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. The skilled artisan can produce variants having single or multiple amino acid substitutions, deletions, additions, or replacements. These variants may include, inter alia: (a) variants in which one or more amino acid residues are substituted with conservative or non-conservative amino acids, (b) variants in which one or more amino acids are added to the polypeptide or protein, (c) variants in which one or more of the amino acids includes a substituent group, and (d) variants in which the polypeptide or protein is fused with another polypeptide such as serum albumin. The techniques for obtaining these variants, including genetic (suppressions, deletions, mutations, etc.), chemical, and enzymatic techniques, are known to persons having ordinary skill in the art. A variant polypeptide preferably comprises at least about 14 amino acids.

A “heterologous protein” refers to a protein not naturally produced in the cell.

A “mature protein” refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

The term “signal peptide” refers to an amino terminal polypeptide preceding the secreted mature protein. The signal peptide is cleaved from and is therefore not present in the mature protein. Signal peptides have the function of directing and translocating secreted proteins across cell membranes. Signal peptide is also referred to as signal protein.

A “signal sequence” is included at the beginning of the coding sequence of a protein to be expressed on the surface of a cell. This sequence encodes a signal peptide, N-terminal to the mature polypeptide that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

The term “homology” refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single-stranded-specific nuclease(s) and size determination of the digested fragments.

Accordingly, the term “sequence similarity” in all its grammatical forms refers to the degree of identity or correspondence between nucleic acid or amino acid sequences of proteins that may or may not share a common evolutionary origin (see Reeck et al., 1987, Cell 50:667). However, in common usage and in the instant application, the term “homologous,” when modified with an adverb such as “highly,” may refer to sequence similarity and not a common evolutionary origin.

As used herein, the term “homologous” in all its grammatical forms and spelling variations refers to the relationship between proteins that possess a “common evolutionary origin,” including proteins from superfamilies and homologous proteins from different species (Reeck et al., supra). Such proteins (and their encoding genes) have sequence homology, as reflected by their high degree of sequence similarity.

In a specific embodiment, two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Sambrook et al., 1989, supra.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

A “substantial portion” of an amino acid or nucleotide sequence comprises enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence.

The term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences was performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=S and DIAGONALS SAVED=5.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

Methods of the Invention

Applicant's invention provides methods to express two or more genes of interest in a host cell. More specifically, this invention provides methods to reduce or eliminate transcriptional interference between two or more tandemly arranged genes or gene expression cassettes in a host cell. The methods according to the invention may be used to express two or more expression cassettes to produce two or more desired polypeptides within a host cell.

In a specific embodiment, the present invention relates to a method to reduce or eliminate transcriptional interference between two tandemly arranged genes or gene expression cassettes in a host cell comprising introducing into the host cell a polynucleotide comprising a first gene expression cassette encoding a first polypeptide, and a second gene expression cassette encoding a second polypeptide, whereby the first gene expression cassette and the second gene expression cassette are positioned in a tandem orientation; and culturing the host cell under conditions, whereby transcriptional interference between the first gene expression cassette and the second gene expression cassette is reduced or eliminated and the first polypeptide and the second polypeptide are expressed.

In a specific embodiment, the present invention relates to a method to reduce or eliminate transcriptional interference between two tandemly arranged genes or gene expression cassettes in a host cell comprising introducing into the host cell a polynucleotide comprising a first gene expression cassette encoding a first polypeptide, and a second gene expression cassette encoding a second polypeptide, whereby the first gene expression cassette and the second gene expression cassette are positioned in a head (5′)-to-head (5′) orientation relative to each other; and culturing the host cell under conditions, whereby transcriptional interference between the first gene expression cassette and the second gene expression cassette is reduced or eliminated and the first polypeptide and the second polypeptide are expressed.

In a specific embodiment, the present invention relates to a method to reduce or eliminate transcriptional interference between two tandemly arranged genes or gene expression cassettes in a host cell comprising introducing into the host cell a polynucleotide comprising a) a first gene expression cassette encoding a first polypeptide, b) a spacer polynucleotide, and c) a second gene expression cassette encoding a second polypeptide, whereby the first gene expression cassette and the second gene expression cassette are positioned in a tandem orientation and the spacer polynucleotide of b) is positioned between the first expression cassette and the second expression cassette; and culturing the host cell under conditions, whereby transcriptional interference between the first gene expression cassette and the second gene expression cassette is reduced or eliminated and the first polypeptide and the second polypeptide are expressed.

In another specific embodiment, the present invention relates to a method to reduce or eliminate transcriptional interference between two or more tandemly arranged genes or gene expression cassettes in a host cell comprising introducing into the host cell a polynucleotide comprising a) a first gene expression cassette, b) a spacer polynucleotide, and c) a second gene expression cassette, whereby the first gene expression cassette and the second gene expression cassette are positioned in a tail (3′)-to-tail (3′) orientation relative to each other and the spacer polynucleotide of b) is positioned between the first expression cassette and the second expression cassette; and culturing the host cell under conditions, whereby transcriptional interference between the first gene expression cassette and the second gene expression cassette is reduced or eliminated and the first polypeptide and the second polypeptide are expressed. In a preferred embodiment, the spacer polynucleotide of b) is a transcription blocker polynucleotide comprising a nucleic acid sequence of SEQ ID NO: 1.

In another specific embodiment, the present invention relates to a method to reduce or eliminate transcriptional interference between two or more tandemly arranged genes or gene expression cassettes in hots cell comprising introducing into the host cell a polynucleotide comprising a) a first gene expression cassette, b) a spacer polynucleotide, and c) a second gene expression cassette, whereby the first gene expression cassette and the second gene expression cassette are positioned in a head (5′)-to-tail (3′) orientation relative to each other and the spacer polynucleotide of b) is positioned between the first expression cassette and the second expression cassette; and culturing the host cell under conditions, whereby transcriptional interference between the first gene expression cassette and the second gene expression cassette is reduced or eliminated and the first polypeptide and the second polypeptide are expressed. In a preferred embodiment, the spacer polynucleotide of b) is a transcription blocker polynucleotide comprising a nucleic acid sequence of SEQ ID NO: 1.

In another specific embodiment, the present invention relates to a method to reduce or eliminate transcriptional interference between two or more tandemly arranged genes or gene expression cassettes in a host cell comprising introducing into the host cell a polynucleotide comprising a) a first gene expression cassette, b) a spacer polynucleotide comprising at least a 40% adenine and thiamine nucleotide content, and c) a second gene expression cassette, whereby the first gene expression cassette and the second gene expression cassette are positioned in a head (5′)-to-tail (3′) orientation relative to each other and the spacer polynucleotide of b) is positioned between the first expression cassette and the second expression cassette; and culturing the host cell under conditions, whereby transcriptional interference between the first gene expression cassette and the second gene expression cassette is reduced or eliminated and the first polypeptide and the second polypeptide are expressed.

Preferably, the spacer polynucleotide of b) comprises at least a 46% adenine and thiamine nucleotide content. In a specific embodiment, the spacer polynucleotide of b) comprising at least a 46% adenine and thiamine nucleotide content comprises a polynucleotide sequence comprising SEQ ID NO: 2 (702 bp BstEII lambda fragment).

More preferably, the spacer polynucleotide of b) comprises at least a 48% adenine and thiamine nucleotide content. In a specific embodiment, the spacer polynucleotide of b) comprising at least a 48% adenine and thiamine nucleotide content comprises a polynucleotide sequence comprising SEQ ID NO: 3 (1519 bp StuI lambda fragment).

Even more preferably, the spacer polynucleotide of b) comprises at least a 63% adenine and thiamine nucleotide content. In a specific embodiment, the spacer polynucleotide of b) comprising at least a 63% adenine and thiamine nucleotide content comprises a polynucleotide sequence comprising SEQ ID NO: 4 (2322 bp HindIII lambda fragment).

In another specific embodiment, the present invention relates to a method to reduce or eliminate transcriptional interference between two or more tandemly arranged genes or gene expression cassettes in a host cell comprising introducing into the host cell a polynucleotide comprising a) a first gene expression cassette, b) a spacer polynucleotide, and c) a second gene expression cassette, whereby the first gene expression cassette and the second gene expression cassette are positioned in a head (5′)-to-tail (3′) orientation relative to each other and the spacer polynucleotide of b) is positioned between the first expression cassette and the second expression cassette; and culturing the host cell under conditions, whereby transcriptional interference between the first gene expression cassette and the second gene expression cassette is reduced or eliminated and the first polypeptide and the second polypeptide are expressed. Preferably, the spacer polynucleotide of b) comprises a polynucleotide sequence comprising SEQ ID NO: 2 (702 bp BstEII lambda fragment). More preferably, the spacer polynucleotide of b) comprises a polynucleotide sequence comprising SEQ ID NO: 3 (1519 bp StuI lambda fragment). Even more preferably, the spacer polynucleotide of b) comprises a polynucleotide sequence comprising SEQ ID NO: 4 (2322 bp HindIII lambda fragment).

Host Cells of the Invention

As described above, the methods of the present invention may be used to express two or more gene expression cassettes in a host cell. The host cell may be a prokaryotic and eukaryotic host cell, and may be selected from the group consisting of bacterial, fungal, yeast, plant, animal, and mammalian host cells. Examples of bacterial, fungal and yeast host cells include but are not limited to: Aspergillus, Trichoderma, Saccharomyces, Pichia, Candida, Hansenula, Synechocystis, Synechococcus, Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

In a preferred embodiment, this invention provides methods to reduce or eliminate transcriptional interference between two or more genes or gene expression cassettes in a eukaryotic host cell. In a more preferred embodiment, this invention provides methods to reduce or eliminate transcriptional interference between two or more genes or gene expression cassettes in a plant host cell. The plant host cell may be either a monocot or dicot plant cell. In a specific embodiment, the plant host cell is selected from the group consisting of an apple, Arabidopsis, bajra, banana, barley, beans, beet, blackgram, chickpea, chilies, cucumber, eggplant, favabean, maize, melon, millet, mungbean, oat, okra, Panicum, papaya, peanut, pea, pepper, pigeonpea, pineapple, Phaseolus, potato, pumpkin, rice, sorghum, soybean, squash, sugarcane, sugarbeet, sunflower, sweet potato, tea, tomato, tobacco, watermelon, and wheat host cell.

The present invention also relates to a transformed host cell that is produced as a result of a method of the invention. Thus, the present invention relates to a transformed host cell comprising two or more gene expression cassettes resulting from Applicant's methods. Preferably, the transformed host cell is selected from the group consisting of an apple, Arabbidopsis, bajra, banana, barley, beans, beet, blackgram, chickpea, chilies, cucumber, eggplant, favabean, maize, melon, millet, mungbean, oat, okra, Panicum, papaya, peanut, pea, pepper, pigeonpea, pineapple, Phaseolus, potato, pumpkin, rice, sorghum, soybean, squash, sugarcane, sugarbeet, sunflower, sweet potato, tea, tomato, tobacco, watermelon, and wheat host cell.

Plant Cell Transformation and Gene Expression

Gene expression in transgenic plant host cells or transgenic plants is useful for production of various desirable traits or gene products, including but not limited to: control of flowering, herbicide resistance, fungicide resistance, insecticide resistance, plant size or form, nutrient content, drought-tolerance, and the like; various pathway intermediates for the modulation of pathways already existing in the host for the synthesis of new products heretofore not possible using the host; examination and determination of ectopic gene expression and gene function, particularly as related to the fields of genomics and proteomics. Additionally, the gene products may be useful for conferring higher growth yields of the host or for enabling alternative growth mode to be utilized.

Plant cell transformation is well known in the art (see Trigiano and Gray, 2^{nd} edition, 2000, CRC press, New York; Gartland and Davey, 1995, Humana Press, Totowa, N.J.; Maliga et al., 995, Cold Spring Harbor Lab Press, New York; and Sambrook et al., 1989, Cold Spring Harbor Lab Press, New York) and may be achieved by various methods including but not limited to: electroporation, Agrobacterium-mediated, particle bombardment, and the like. Expression of desired gene products involves growing the transformed host cells under conditions that induce expression of the transformed genes or gene expression cassettes. The cells are harvested and gene products are isolated according to protocols specific for the gene product.

In a specific embodiment of the present invention, the transformed host cell is an apple, Arabidopsis, bajra, banana, barley, beans, beet, blackgram, chickpea, chilies, cucumber, eggplant, favabean, maize, melon, millet, mungbean, oat, okra, Panicum, papaya, peanut, pea, pepper, pigeonpea, pineapple, Phaseolus, potato, pumpkin, rice, sorghum, soybean, squash, sugarcane, sugarbeet, sunflower, sweet potato, tea, tomato, tobacco, watermelon, or wheat cell.

Genes of Interest

Nucleic acid sequence information for a desired protein can be located in one of many public access databases, for example, GENBANK, EMBL, Swiss-Prot, and PIR, or in many biology related journal publications. Thus, those skilled in the art have access to nucleic acid sequence information for virtually all known genes. Such information can then be used to construct the desired constructs for the insertion of the gene of interest within the expression cassettes used in Applicant's methods described herein.

In a specific embodiment of the invention, at least one of the gene expression cassettes comprises a homologous gene with respect to the host cell. In another specific embodiment of the invention, at least one of the gene expression cassettes comprises a heterologous gene with respect to the host cell. Examples of genes of interest for expression in a host cell using Applicant's methods include, but are not limited to: antigens produced in plants as vaccines, enzymes like alpha-amylase, phytase, glucanes, and xylanse, genes for resistance against insects, nematodes, fungi, bacteria, viruses, and abiotic stresses, nutraceuticals, pharmaceuticals, vitamins, genes for modifying amino acid content, herbicide resistance, cold, drought, and heat tolerance, industrial products, oils, protein, carbohydrates, antioxidants, male sterile plants, flowers, fuels, and other output traits, and the like.

Measuring Gene Expression/Transcription

One useful measurement in Applicant's methods of the invention is that of the transcriptional state of the cell which includes the identities and abundance of RNA, preferably mRNA species. Such measurements are conveniently conducted by measuring cDNA abundance by any of several existing gene expression technologies well known in the art.

Another biological pathway response measurement is by determining the translation state of the cell by measuring the abundances of the constituent protein species present in the cell using processes well known in the art.

Where identification of genes associated with various physiological functions is desired, an assay may be employed in which changes in such functions as cell growth, apoptosis, senescence, differentiation, adhesion, binding to a specific molecules, binding to another cell, cellular organization, organogenesis, intracellular transport, transport facilitation, energy conversion, metabolism, myogenesis, mneurogenesis, and/or hematopoiesis is measured.

Other methods to detect the products of gene expression are known including Southern blots (DNA detection), dot or slot blots (DNA, RNA), Northern blots (RNA), and RT-PCR (RNA). Although less preferred, labeled proteins can be used to detect a particular nucleic acid sequence to which it hybridizes.

In some cases it is necessary to amplify the amount of a nucleic acid sequence. This may be carried out using one or more of a number of suitable methods including, for example, polymerase chain reaction (“PCR”), reverse-transcription polymerase chain reaction (“RT-PCR”), ligase chain reaction (“LCR”), strand displacement amplification (“SDA”), transcription-based amplification, and the like. PCR is carried out in accordance with known techniques in which, for example, a nucleic acid sample is treated in the presence of a heat stable DNA polymerase, under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present.

Nucleic acid array technology is a useful technique for determining differential mRNA expression. This type of analysis may be desirable when analyzing gene function or downstream pathway effects of gene expression. Such technology includes, for example, oligonucleotide chips and DNA microarrays. These techniques rely on DNA fragments or oligonucleotides which correspond to different genes or cDNAs which are immobilized on a solid support and hybridized to probes prepared from total mRNA pools extracted from cells, tissues, or whole organisms and converted to cDNA. Oligonucleotide chips are arrays of oligonucleotides synthesized on a substrate using photolithographic techniques. Chips have been produced which can analyze up to 1700 genes. DNA microarrays are arrays of DNA samples, typically PCR products, that are robotically printed onto a microscope slide. Each gene is analyzed by a full or partial-length target DNA sequence. Microarrays with up to 10,000 genes are now routinely prepared commercially. The primary difference between these two techniques is that oligonucleotide chips typically utilize 25-mer oligonucleotides which allow fractionation of short DNA molecules whereas the larger DNA targets of microarrays, approximately 1000 base pairs, may provide more sensitivity in fractionating complex DNA mixtures.

The present invention may be better understood by reference to the following non-limiting Examples, which are provided as exemplary of the invention.

### EXAMPLE 1

This Example describes a method for reducing or eliminating transcriptional interference between two tandemly arranged gene expression cassettes. Applicant has herein examined the influence of a gene on the expression of an adjacent gene. Briefly, two gene expression cassettes were cloned in all possible orientations to study the effect of orientation of one gene on the expression of the other gene. Applicant has now shown that transcriptional interference is orientation dependent and can be reduced or eliminated depending upon the arrangement of the gene expression cassettes in relation to each other or by the insertion of spacer polynucleotides between the gene expression cassettes.

General Methods

Standard recombinant DNA and molecular cloning techniques used herein are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Methods for plant tissue culture, transformation, plant molecular biology, and plant, general molecular biology may be found in “Plant Tissue Culture Concepts and Laboratory Exercises” edited by R N Trigiano and D J Gray, 2^{nd} edition, 2000, CRC press, New York; “Agrobacterium Protocols” edited by KMA Gartland and M R Davey, 1995, Humana Press, Totowa, N.J.; “Methods in Plant Molecular Biology” P. Maliga et al., 995, Cold Spring Harbor Lab Press, New York; and “Molecular Cloning” J. Sambrook et al., 1989, Cold Spring Harbor Lab Press, New York.

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, DC. (1994) or by Thomas D. Brock in Biotechnology: A Textbook of Industrial Microbiology, Second Edition, Sinauer Associates, Inc., Sunderland, Mass. (1989). All reagents, restriction enzymes and materials used for the growth and maintenance of host cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Manipulations of genetic sequences may be accomplished using the suite of programs available from the Genetics Computer Group Inc. (Wisconsin Package Version 9.0, Genetics Computer Group (GCG), Madison, Wis.). Where the GCG program “Pileup” is used the gap creation default value of 12, and the gap extension default value of 4 may be used. Where the CGC “Gap” or “Bestfit” program is used the default gap creation penalty of 50 and the default gap extension penalty of 3 may be used. In any case where GCG program parameters are not prompted for, in these or any other GCG program, default values may be used.

The meaning of abbreviations is as follows: “h” means hour(s), “min” means minute(s), “sec” means second(s), “d” means day(s), “μl” means microliter(s), “ml” means milliliter(s), “L” means liter(s), “μM” means micromolar, “mM” means millimolar, “μg” means microgram(s), “mg” means milligram(s), “A” means adenine or adenosine, “T” means thymine or thymidine, “G” means guanine or guanosine, “C” means cytidine or cytosine, “x g” means times gravity, “nt” means nucleotide(s), “aa” means amino acid(s), “bp” means base pair(s), “kb” means kilobase(s), “k” means kilo, “μ” means micro, “%” means percent, and “°C.” means degrees Celsius.

1.1 Plasmid Constructs

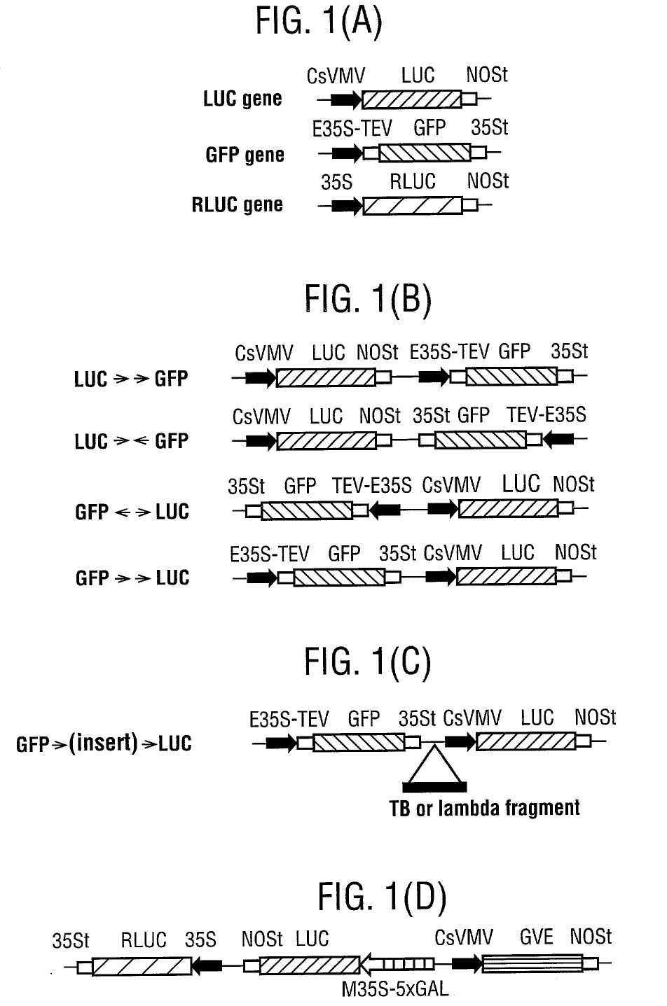

Genes for firefly Photinus pyralis luciferase (LUC) and jellyfish Aequorea vactoria green fluorescent protein (GFP) suitable for expression in plant cells were constructed in pBluescriptII SK—using standard procedures (15). As illustrated in FIG. 1A, the LUC gene construct contained the cassava vein mosaic virus (CsVMV) promoter (17), LUC coding sequence, and NOS terminator sequence. The GFP gene construct contained a duplicated cauliflower mosaic virus 35S promoter (E35S), tobacco etch virus (TEV) enhancer, GFP coding sequence, and 35S terminator sequence (14). As an internal control for protoplast assays, a Renilla Luciferase (RLUC) gene was constructed with a 35S promoter and NOS terminator. To obtain plasmids containing both genes in all possible orientations, the GFP gene was cloned before or after the LUC gene in four possible orientations (FIG. 1B). In addition, a 154 bp mammalian transcription blocker (TB) sequence (5, SEQ ID NO: 1, GenBank accession number U89937), a 702 BstE II fragment (SEQ ID NO: 2), a 1519 Stu I fragment (SEQ ID NO: 3), or a 2322 bp Hind III fragment (SEQ ID NO: 4) from λ phage DNA (GenBank accession number J02459) was cloned between the LUC and GFP gene cassettes (FIG. 1C). An ecdysone receptor (EcR)-based, chemically inducible gene expression plasmid was constructed by cloning the chimeric GVE gene cassette (CsVMV promoter—GAL4 DNA binding domain:VP16 activation domain:EcR ligand binding domain—NOS terminator), inducible LUC gene cassette (5×GAL4 response element—minimal 35S promoter—LUC coding sequence—35S terminator), and constitutive RLUC gene cassette (35S promoter—RLUC coding sequence—35S terminator) in the orientation shown in FIG. 1D. All plasmid DNAs were prepared using Qiagen Plasmid Maxi kit.

1.2 Protoplast Transfections

Protoplasts were prepared from actively growing tobacco BY-2 suspension cells (Japan Tobacco, Japan) and cultured essentially as described in Kikkawa et al. (9). One ml of protoplasts (5×10⁵ to 1×10⁶ cells) was mixed with plasmid DNA and electroporated at 0.56 K Volts for 80 μ seconds using an electroporation system with petripulser electrode (BTX). Equimolar plasmid DNA was added to transfections and the amount of DNA added varied from 23 to 30 μg depending on the plasmid size. To standardize the luciferase enzyme assays, 2 μg of the RLUC plasmid DNA were also added to each transfection as an internal control. Following the electroporation, protoplasts were diluted with 1 ml of 2×protoplast culture medium, aliquoted as two 1 ml cultures and incubated at 27° C. for 16-17 h. Each plasmid construct was tested in 5 to 7 independent transfection experiments.

1.3 Luciferase Enzyme Assay

Protoplast cultures were lysed by adding 250 μl of 5× passive lysis buffer (Promega) and shaking at room temperature for 10 minutes. A 20 μl lysate from each culture (two assays per transfection) were assayed for luciferase (Luc) and Renilla luciferase (RLuc) enzyme activities using the Dual-Luciferase Reporter Assay Kit (Promega) according to the manufacturer's suggestions. The assay was performed with a luminometer plate reader (Dynex). The luciferase enzyme activities were measured and expressed as ratios of luciferase to Renilla luciferase activities in relative light units.

1.4 Ecdysone Receptor-Based Gene Induction

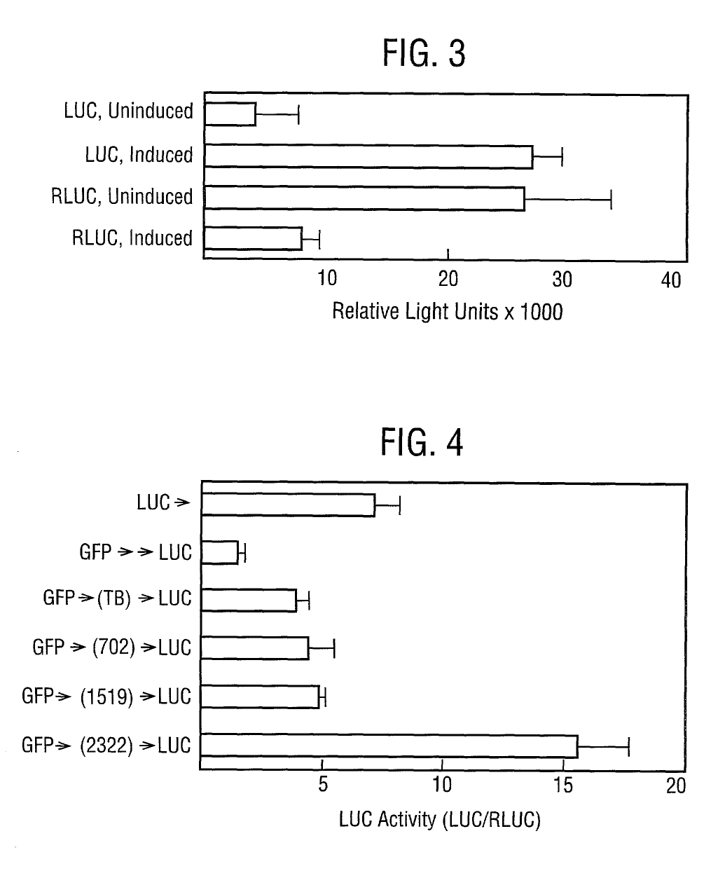

Ecdysone receptor (EcR)-based gene induction plasmid was constructed (as described in Example 1.1, see FIG. 1D) by cloning 1) a constitutively expressed receptor cassette comprising a CsVMV promoter, operably linked to a GVE (GAL4 DNA binding domain: VP16 activation domain:EcR ligand binding domain) encoding polynucleotide and a NOS terminator polynucleotide and 2) an inducible reporter gene cassette comprising 5×GAL binding sites (response elements) operably linked to a minimal 35S promoter operably linked to a Luc encoding polynucleotide and a 35S terminator polynucleotide in a head-to-head orientation. A third expression cassette comprising a 35S promoter operably linked to a Renilla Luc encoding polynucleotide and a 35S terminator polynucleotide was then cloned downstream of the Luc gene expression cassette (see FIGS. 1D and 3). Induction of the Luc gene expression cassette was controlled by the addition of EcR ligand methoxyfenozide.

1.5 RESULTS AND DISCUSSION

Results are summarized in the following Tables 1 and 2.

TABLE 1

| | Luc/RLuc ratio | |
|---|---|---|
| | Averages and (values) of 2 to 7 | Relative |
| Plasmid | transfections | to Luc→ |
| Luc→ | 7.06 (7.16, 5.38, 6.99. 6.84, 8.63, 6.56, | 100 |
| | 7.85) | |
| GFP← →Luc | 10.05 (10.48, 10.59, 9.96, 10.62, | 142 |
| | 10.76, 7.87) | |
| Luc→ ←GFP | 3.31 (3.17, 3.14, 3.53, 3.58, 3.15) | 47 |
| GFP→ →Luc | 1.41 (1.41, 1.39, 1.32, 1.40, 1.50, 1.43) | 20 |
| Luc→ →GFP | 7.38 (7.26, 6.95, 7.69, 7.56, 7.45) | 105 |
| (TB) Luc→ | 7.76 (8.20, 7.16, 8.67, 8.39, 6.38) | 110 |
| →GFP | | |
| Luc→ (TB) | 8.76 (10.33, 9.53, 9.55, 7.06, 7.37) | 123 |
| ←GFP | | |
| GFP→ (TB) | 3.96 (3.82, 3.76, 3.09, 4.01, 4.26, 4.83) | 56 |
| →Luc | | |
| GFP← (TB) | 11.73 (11.62, 13.24, 11.36, 11.17, | 166 |
| Luc→ | 13.3, 9.57) | |
| GFP→ (2.3 kb) | 14.81 (14.68, 14.95) | 209 |
| →Luc | | |
| GFP→ | 0.0003 (0.0003, 0.0004) | 0.04 |

TABLE 2

| | Luc | Luc | RLuc | RLuc |
|---|---|---|---|---|
| Construct | Uninduced | Induced | Uninduced | Induced |
| ←RLuc Luc← | 1,902 | 25,428 | 30,588 | 8,832 |
| →GVE | | | | |

To determine the transcriptional interference on the expression of one gene from its adjacent gene, two genes (promoter—coding sequence—terminator) were cloned in all four possible orientations (FIG. 1B). The promoters used for the expression of GFP and LUC in plant cells were cauliflower mosaic virus 35S (35S) and cassava vein mosaic virus (CsVMV) promoters. Both 35S and CsVMV promoters are commonly used to express transgenes in plant cells. Both the duplicated 35S promoter (E35S) and the CsVMV promoter showed similar promoter strength in tobacco BY-2 protoplasts in our previous experiments (data not shown). Specifically, the E35Sp-Luc-NOSt construct and CsVMVp-Luc-NOSt construct, when evaluated on separate plasmids have similar Luc activity showing that the two promoters used to express GFP and Luc in this study have similar strength (data not shown).

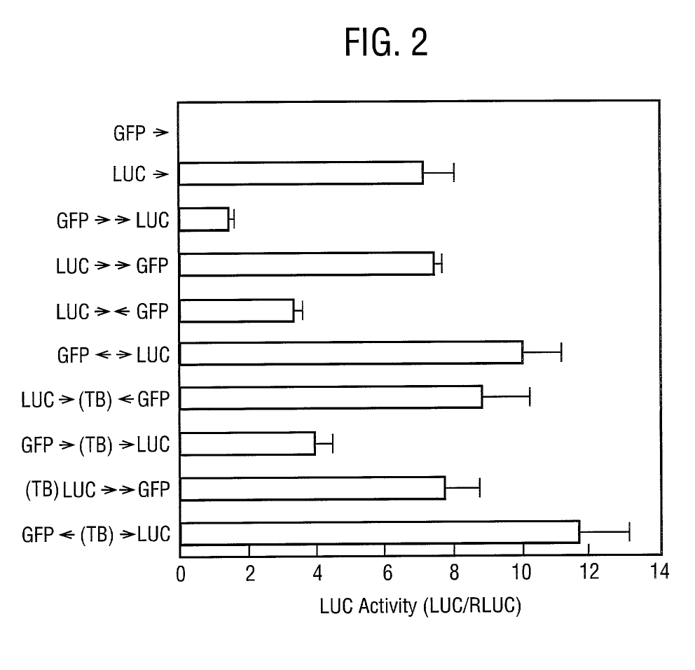

No endogenous LUC enzyme activity was detected in tobacco BY-2 protoplasts. LUC enzyme activity was about 1 relative light unit (RLU) with a LUC/RLUC ratio of 0.0003 in protoplasts transfected with salmon sperm DNA or GFP gene (GFP→). In contrast, protoplasts transfected with the LUC gene (LUC→) showed LUC activities of about 50,000 RLUs with an average LUC/RLUC ratio of 7.07 (FIG. 2). When the GFP gene was cloned upstream of the LUC gene in a head-to-tail orientation (GFP→→LUC), the LUC activity was reduced by ˜80%. However, no inhibitory interference was detected on LUC gene expression when the GFP gene was downstream of the LUC gene in the head-to-tail (LUC→→GFP) orientation (FIG. 2). The LUC activity was reduced by 53% when GFP and LUC genes were in tail-to-tail (Luc→←GFP) orientation. There was no decrease in LUC gene expression when the orientation was head-to-head (GFP←→LUC).

In order to test if the activation of an upstream promoter can cause transcriptional interference on downstream gene expression, an ecdysone receptor (EcR) based, chemically inducible gene expression system was used (unpublished). This system is similar to a glucocorticoid receptor based, dexamethasone-inducible gene expression system developed for plants by Aoyama and Chua (1). It contains a constitutively expressed chimeric GVE receptor gene (CsVMV promoter—sequences coding for GAL4 DNA binding domain, VP16 activation domain, and EcR ligand binding domain—NOS terminator) and an inducible promoter (five copies of GAL4 response element+minimal 35S promoter) to drive LUC gene expression (FIG. 1D). When the ligand methoxyfenozide (4) is applied to protoplasts, it binds to and activates the chimeric receptor. The activated receptor then induces LUC gene expression. This system was used to activate the upstream LUC gene, and the influence of LUC activation on the downstream constitutive RLUC gene expression was then measured. After the addition of a ligand to protoplasts, the LUC gene expression increased from 1,902 to 25,428 relative light units (RLU), while the RLUC expression decreased from 30,588 to 8832 RLU (FIG. 3). Therefore, activation of the upstream Luc gene with ecdysone receptor-mediated inducible gene expression system reduced the expression of downstream, constitutively expressed RLuc expression by 71%. This result reinforces the result obtained with the head-to-tail GFP→→Luc construct.

The above results show that the LUC gene expression was affected by the location and the orientation of adjacent GFP gene, and induction of the upstream promoter reduced the downstream gene expression. Neither the 35S nor the NOS terminator was sufficient in preventing the transcriptional interference between tandem genes. In an earlier study (8), the interference was blocked by placing a terminator (polyadenylation) sequence on either side of the transgene. In the construct GFP→→LUC (FIG. 2), LUC activity was reduced 80% even though there were terminators before the LUC gene and after LUC coding sequence. It is possible that the strength of the promoter is important in determining the degree of transcriptional interference. The results described herein suggest that the transcriptional interference between the GFP and LUC genes was caused by RNA polymerase read-through and/or by antisense RNA production. The interference is probably not due to the local depletion of diffusible transcription factors or DNA topological constraints since the LUC gene expression was not reduced by the expression of GFP in all orientations.

Applicant's results demonstrate that transcriptional interference can be avoided by cloning two genes in the head-to-head (←→) orientation. However, it is not possible to have head-to-head orientation when 3 or more genes are cloned in tandem. Also, the integration of a transgene into plants is random and adjacent genes on the chromosome can cause interference. It was reported in mammalian cells that poly(A) signal and a downstream transcriptional pause site are required for transcriptional termination by RNA polymerase II. Placing a poly(A) signal, a transcription pause site or both between tandem genes blocked the transcriptional interference (5). On this basis, an additional nucleotide sequence was placed after the upstream gene terminator to examine the effect on transcriptional interference on a downstream gene expression. A mammalian transcription blocker (TB) sequence was cloned between the GFP and LUC genes in head-to-tail (GFP→(TB)→LUC) and tail-to-tail (LUC→(TB)←GFP) orientations (FIG. 1C). The TB sequence (SEQ ID NO: 1) is a 154 bp DNA fragment containing a synthetic poly(A) site (nucleotides 1-49 of SEQ ID NO: 1) and a transcription pause site from human alpha 2 globin gene (5; nucleotides 63-154 of SEQ ID NO: 1). As shown in FIG. 2, transcriptional interference was completely blocked by the TB sequence in the construct LUC→(TB)←GFP and partially blocked in the construct GFP→(TB)→LUC. As expected, cloning the TB sequence before the LUC gene in the construct (TB)LUC→→GFP and between the LUC and GFP genes in the construct LUC←(TB)→GFP did not increase the LUC activity as there was no interference in these orientations (FIG. 2).

The effect of other DNA fragments to eliminate or reduce the transcriptional interference in the construct GFP→→SLUC was also examined. Three different DNA fragments from λ phage [702 bp BstE II (SEQ ID NO: 2), 1519 bp Stu I (SEQ ID NO: 3), or 2322 bp Hind III (SEQ ID NO: 4) fragments] were tested by cloning them between the GFP and LUC genes. As shown in FIG. 4, the 702 bp and 1519 bp fragments partially negated the interference. The LUC/RLUC ratio for the construct GFP→→SLUC increased from 1.41 to 4.42 when a 702 bp λ DNA fragment was inserted (GFP→(702)→LUC) and to 4.88 when a 1519 bp fragment was inserted (GFP→(1519)→LUC). Insertion of a 2322 bp λ DNA fragment between GFP and LUC genes (GFP→(2322)→LUC) not only eliminated the interference but also increased the LUC expression when compared to the construct LUC→(FIG. 4). The 702 bp and 1519 bp λ DNA fragments have 46% and 48% AT content, respectively. In contrast, the 2322 bp fragment has 63% AT content. AT rich sequences can act as transcription termination signals and the elimination of interference by the 2322 bp fragment may be due to its higher AT content and/or longer length.

The experiments described herein show that the transcriptional interference between adjacent genes can be significant and that this interference can be eliminated by cloning two genes in head-to-head (←→) orientation. When this orientation is not possible, particularly when three or more genes or gene expression cassettes are cloned, transcriptional interference can be reduced or eliminated by placing a spacer polynucleotide, such as a TB polynucleotide or a lambda phage polynucleotide, between the genes or cassettes.

### EXAMPLE 2

This Example describes an ecdysone receptor-based chemical-inducible system for plant gene regulation. Applicant has developed an inducible gene expression system with potential for field application using the spruce budworm Choristeneuria fumiferana (Cf) ecdysone receptor (EcR) and non-steroidal ecdysone agonists. Chimeric transcription factors were made using different DNA binding and activation domains and an EcR ligand binding domain. A reporter gene luciferase was cloned downstream of a DNA element where this chimeric transcription factor can bind. This chimeric transcription factor does not activate transcription in the absence of a ligand. Addition of the ligand methoxyfenozide, which has exceptional health and environmental safety profiles, induced luciferase expression. Applicant has used this chemical-inducible system in transient assays with tobacco protoplasts and in transgenic Arabidopsis and tobacco plants. Applicant's results show that the system based on the EcR ligand binding domain is very effective and can be used to express high levels of protein.

Introduction: An inducible system to activate or inactivate plant gene expression has many potential applications in basic understanding of gene function, in manipulating complex developmental pathways, and in plant biotechnology. Many chemical-inducible systems have been developed for plants (18-20). Systems based on plant promoters whose expression is triggered by chemicals (e.g., salicylic acid) may not be suitable for field application because endogenous chemically inducible promoters may have high basal expression and may respond to environmental and plant signals in addition to applied chemical. Systems based on artificial chemical-induction use components derived from non-plant sources and have many advantages.

The chemical-inducible gene regulation systems comprise two transcription units. The product of the first transcription unit is a activated transcription that is responsive to chemicals. The second transcription unit is under the control of transcription factor and consists of responsive element and minimal promoter where transcription factor can bind. The chemical-inducible systems developed for plants include Tet repressor-based, tetracycline de-repressible (21), tTA-based, tetracycline inactivatable (22), glucocorticoid receptor-based, dexamethasone inducible (1), AlcR-based, ethanol inducible (23), ecdysone receptor (EcR)-based, ecdysone inducible (24), estrogen receptor-based, β-estradiol inducible (25), and tetracycline and glucocorticoid receptors-based, dual control (26). An ideal gene expression system will have the following desirable properties (20): low basal expression levels, high inducibility, specificity to inducer, high dynamic range to inducer concentrations, fast response, switch-off after removal of inducer, and safe inducer.

EcR-Based Plant Gene Regulation System: Growth, molting, and development in insects are regulated by the ecdysone steroid hormone (molting hormone) and the juvenile hormones (4). The molecular target for ecdysone in insects comprises at least an ecdysone receptor (EcR) and an ultraspiracle protein (USP). EcR is a member of the nuclear steroid receptor super family that is characterized by signature DNA and ligand binding domains, and an activation domain (27). Tebufenozide and methoxyfenozide are non-steroidal ecdysone analogs that are marketed worldwide by Rohm and Haas Company as insecticides. Both analogs have exceptional safety profiles to other organisms.

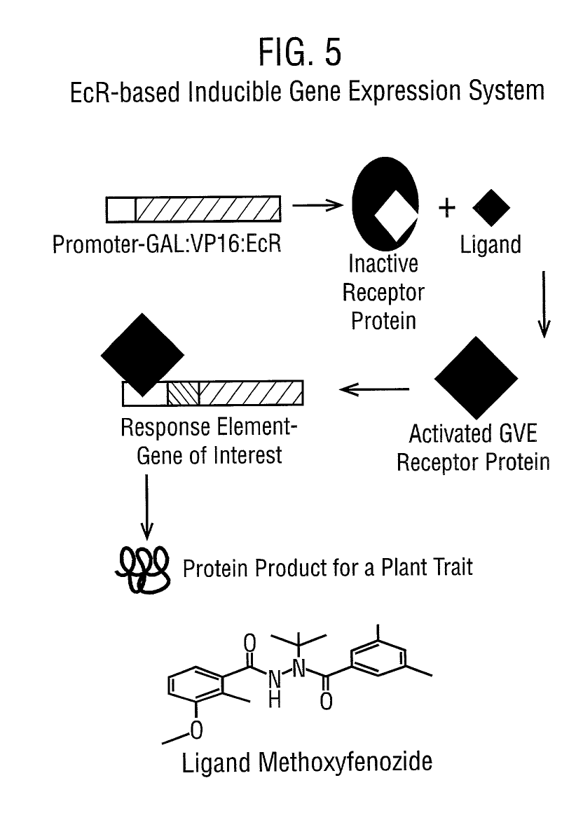

Applicant has developed an inducible gene regulation system suitable for large-scale field application using EcR from spruce budworm and non-steroidal ecdysone agonists (see FIG. 5). The first version of the system comprises a chimeric transcription activator (GVE, GAL4 DNA binding domain, VP16 activation domain, and EcR ligand binding domain) under the control of cassava vein mosaic virus (CsVMV) promoter. The reporter gene luciferase (Luc) is downstream of 5×GAL4 response element and minimal 35S promoter.

Results

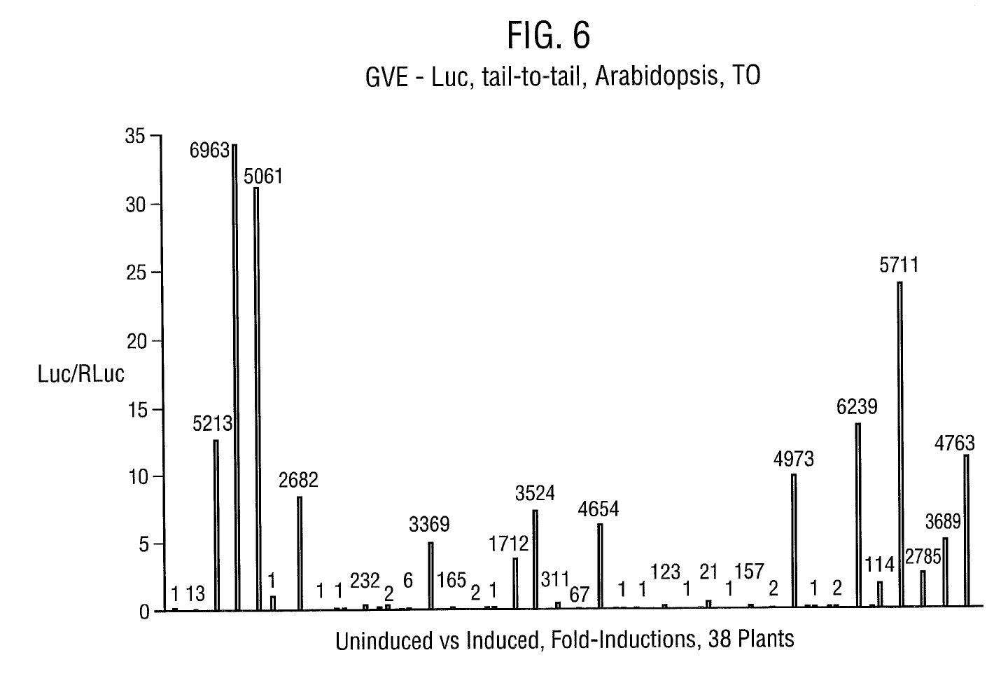

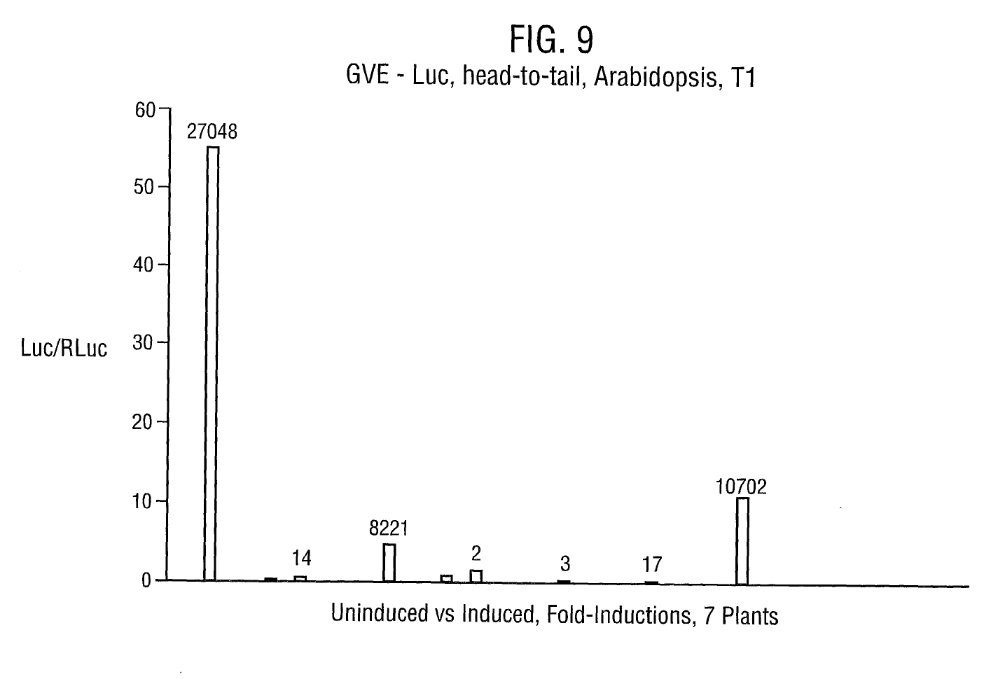

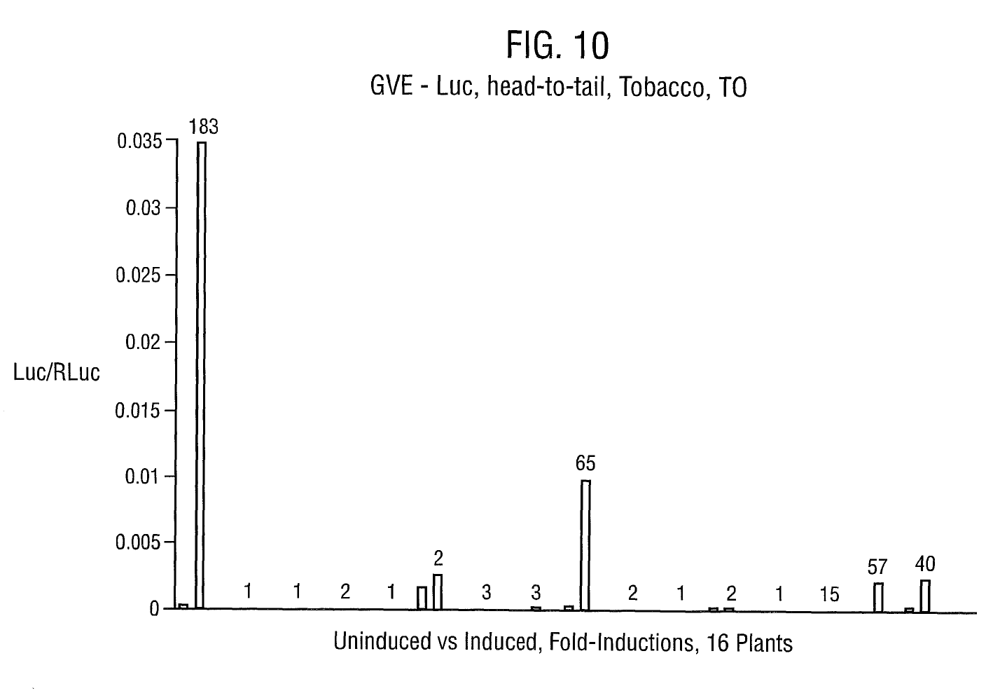

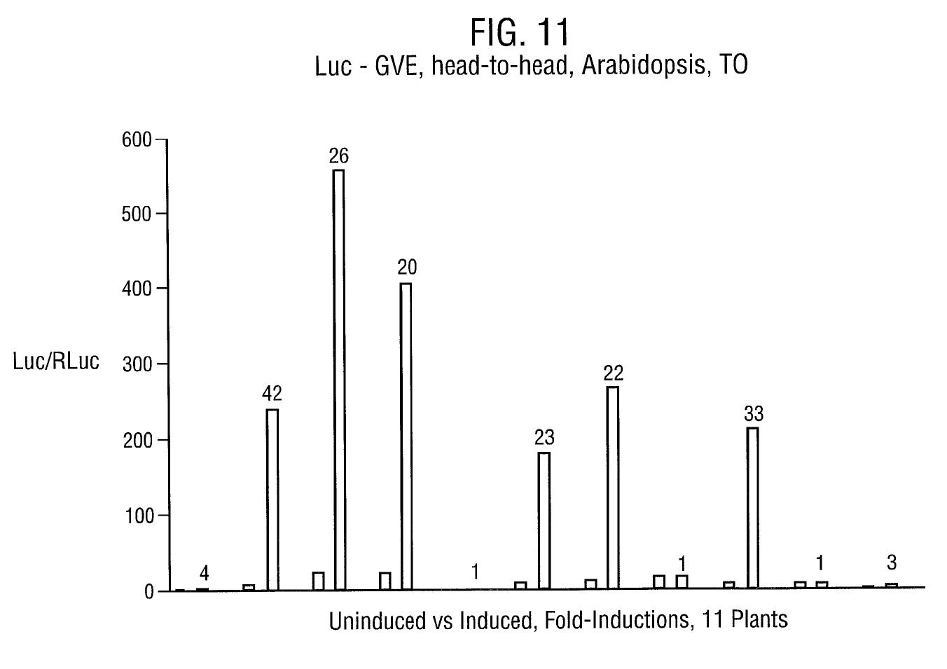

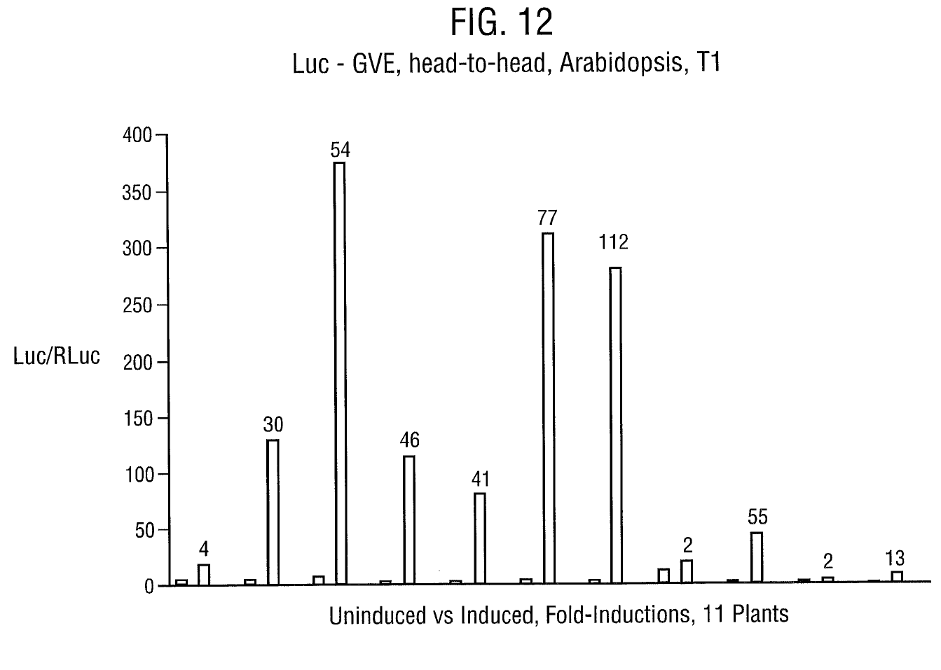

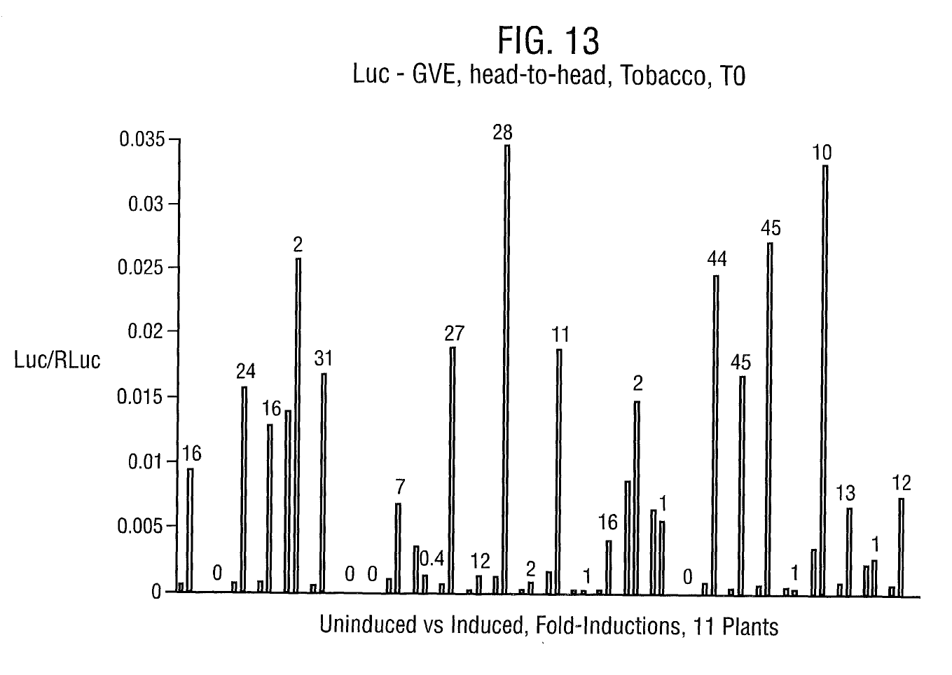

All binary vector constructs have four genes; NOSp-NPTII selectable marker, 35Sp-RLuc constitutive internal control marker, CsVMVp-GVE receptor, and 5×GAL+M35Sp-Luc reporter. The receptor and reporter genes were cloned in all possible orientations; tail-to-tail (GVE→←Luc), head-to-tail (GVE→→Luc and Luc→→GVE), and head-to-head (Lucy←→GVE). Arabidopsis and tobacco plants were transformed by employing standard protocols. Leaf disks taken from T0 plants were incubated in the presence or absence of 10 mM methoxyfenozide for 24 hours and induced Luc and constitutive Rluc activities were assayed using Dual Luciferase Assay Kit from Promega. The Luc activity is presented as the ratio between Luc and Rluc. T1 seed was germinated on agar media containing no or 10 mM methoxyfenozide and assayed for Luc and Rluc activity 3 weeks after germination. Applicant's results are discussed below and are presented in FIGS. 6-13 and in Tables 3-5.

←NOS-NPTII+←35S-RLuc+CsVMV-GVE→+←5×GAL+M35S-Luc

Construct

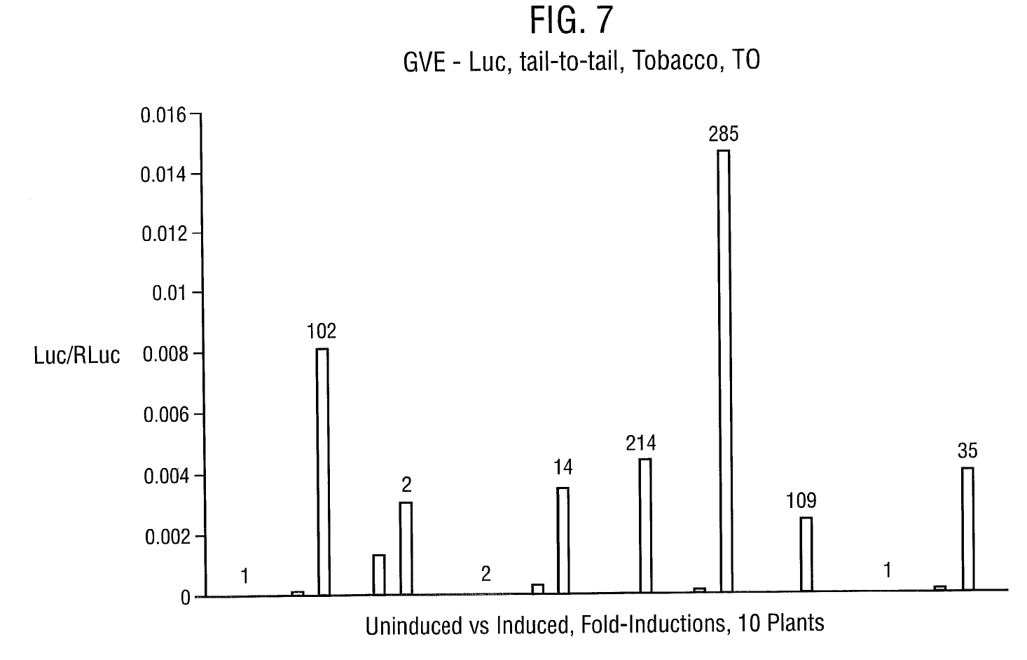

In this construct, the receptor and reporter are in tail-to-tail orientation. Results from Arabidopsis T0 plants are shown in FIG. 6 and results from tobacco T0 plants are shown in FIG. 7.

The uninduced Luc activity in Arabidopsis plants (FIG. 6) ranged from 0.09-89 relative light units (RLU) per mg protein while the induced activities ranged from 2-3558 RLUs/mg. Induced Luc activity in some plants reached to the similar levels as in 35S-Luc plants. Non-transgenic, negative control plants had Luc activities of 0.04-1.02 RLU/mg. Fold-induction varied from 1-6963 and will be higher if Luc background activity value from non-transgenic plants is subtracted. Constitutive RLuc activity, which ranged from 32-2762, was used as an internal control and Luc activity is shown as Luc/RLuc ratio.

←NOS-NPTII+←35S-Rluc+CsVMV-GVE→+←5×GAL+M35S-Luc

Construct

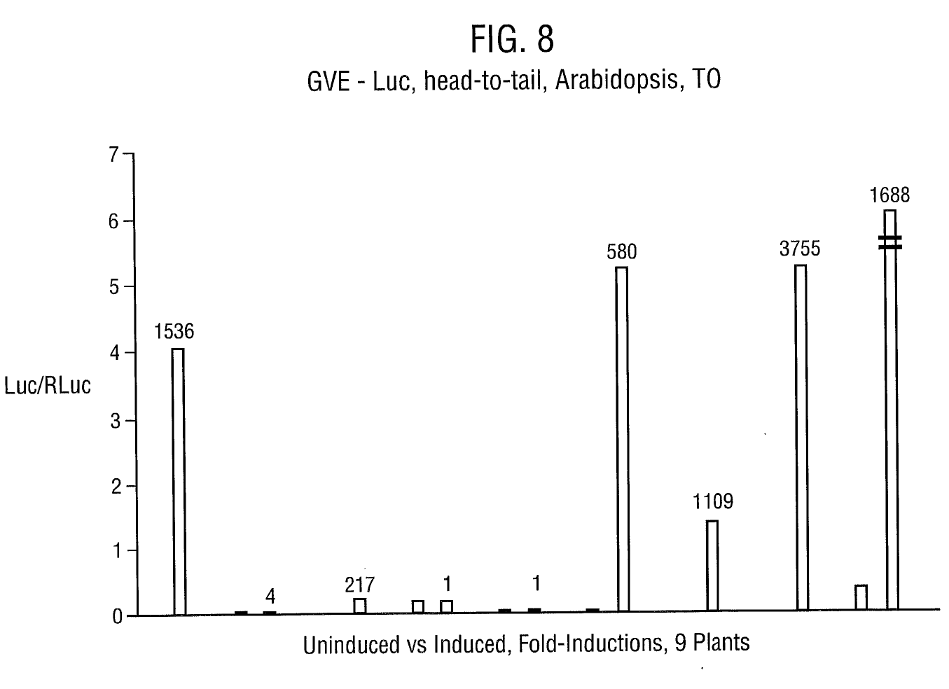

The receptor and reporter are in head-to-tail orientation. Data generated from analyses of this construct in Arabidopsis T0 and T1 plants and Tobacco T0 plants are shown in FIGS. 8-10.

←NOS-NPTII+←35S-Rluc+5×GAL+M35S-Luc←+→CsVMV-GVE

Construct

The reporter and receptor are in head-to-head orientation. Data generated from analyses of this construct in Arabidopsis T0 and T1 plants and Tobacco T0 plants are shown in FIGS. 11-13.

←NOS-NPTII+←35S-Rluc+5×GAL+M35S-Luc←+→CsVMV-GVE

Construct

The reporter and receptor are in head-to-tail orientation. Data generated from analyses of this construct in Tobacco T0 and T1 plants are summarized in Table 3.

TABLE 3
Summary of Reporter/Receptor Constructs in Tobacco T0 and T1 Plants

| | Plants | | Range (Mean) | | Range (Mean) |
|---|---|---|---|---|---|
| Construct | (n) | >3F-I* | LUC, Uninduced | | LUC, Induced |
| GVE→→LUC | 21 | T0: 6 | 0.3-37 | (7) | 3-335 (133) |
| | | T1: 5 | 0.1-7 | (3) | 43-1167 (641) |
| GVE→←LUC | 15 | T0: 9 | 0.1-2 | (1) | 9-220 (67) |
| | | T1: 8 | 0.3-10 | (4) | 6-633 (204) |
| LUC←→GVE | 20 | T0: 12 | 7-107 | (23) | 141-299 (234) |
| | | T1: 11 | 18-210 | (71) | 97-1126 (729) |
| LUC→→GVE | 25 | T0: 14 | 0.4-47 | (12) | 2-530 (258) |
| | | T1: 16 | 0.4-121 | (51) | 56-2433 (1472) |
| VGE→→LUC | 19 | T0: 5 | 0.2-1 | (0.4) | 4-59 (32) |
| | | T1: 7 | 1.8-13 | (7) | 157-489 (295) |
| VGE→←LUC | 26 | T0: 10 | 0.1-8 | (2) | 4-287 (142) |
| | | T1: 13 | 0.1-3 | (1) | 3-940 (330) |
| LUC ←→VGE | 21 | T0: 10 | 0.6-29 | (7) | 14-286 (121) |
| | | T1: 9 | 3-102 | (38) | 102-1738 (832) |

F-I* refers to “fold-induction”

Applicants also tested the effect of the order or arrangement of GAL4, VP 16 and EcR domains within the receptor construct on the uninduced and induced Luc expression levels (see Tables 3-5). Tobacco (BY2) protoplasts were transfected with reporter plasmid and three different receptor plasmids in which the location of VP16 (with respect to other domains) was varied. Protoplasts were incubated in the presence (induced) or absence (uninduced, UI) of 10 mM methoxyfenozide and Luc activity was assayed 17 hours of incubation (see Tables 4 and 5). Higher fold induction was observed for VGE because of reduction in basal activity.

TABLE 4
Protoplast assays with different receptor constructs and reporter on the
same plasmid.

| Receptor | Luc, Uninduced | Luc, Induced | Fold Induction |
|---|---|---|---|
| GVE | 2170 | 29173 | 14 |
| VGE | 516 | 21063 | 43 |
| GEV | 363 | 5802 | 16 |

TABLE 5
Protoplast assays with receptor and reporter on the same plasmid.

| Construct | Luc (RLU) | Fold-Induction |
|---|---|---|
| GVE→ + →Luc | | |
| UI | 2,826 | |
| Induced | 17,334 | 7.63 |
| GVE→ + ←Luc | | |
| UI | 1,786 | |
| Induced | 8,970 | 5.62 |
| Luc→ + →GVE | | |
| UI | 5,941 | |
| Induced | 29,146 | 5.87 |
| Luc← + →GVE | | |
| UI | 10,704 | |
| Induced | 48,365 | 4.90 |

Conclusions: Arabbidopsis and tobacco plants with low uninduced and high induced levels of luciferase were obtained by optimizing receptor (GVE) and reporter gene orientations. The uninduced luciferase activity in some plants was very low (3-5 relative light units compared to 1-3 RLU for control plants). The induced levels in some plants reached the levels of the constitutive 35S-Luc control (data not shown). The fold-induction was increased as high as 27,000 fold.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all base sizes or amino acid sizes, and all molecular weight or molecular mass values, given for nucleic acids or polypeptides are approximate, and are provided for description.

References

1. Aoyama, T. and N.-H. Chua. 1997. Plant J. 11: 605-612.

2. Bhattacharyya, M. K., B. A. Stermer and R. A. Dixon. 1994. Plant J. 6: 957-968.

3. Breyne, P., G. Gheysen, A. Jacobs, M. V. Montagu and A. Depicker. 1992. Mol. Gen. Genet. 235: 389-396.

4. Dhadialla, T. S., G. R. Carlson and D. P. Le. 1998. Annu. Rev. Entomol. 43: 545-569.

5. Eggermont, J. and N. J. Proudfoot. 1993. EMBO J. 12: 2539-2548.

6. Greger, I. H., A. Aranda, and N. J. Proudfoot. 2000. Proc. Natl. Acad. Sci. USA 97: 8415-8420.

7. Grierson, D., R. G. Fray, A. J. Hamilton, C. J. S. Smith, and C. F. Watson. 1991. Trends Biotechnol. 9: 122-123.

8. Ingelbrecht, I., P. Breyne, K. Vancompernolle, A. Jacobs, M. V. Montagu and A. Depicker. 1991. Gene 109: 239-242.

9. Kikkawa, H., T. Nagata, C. Matsui and I. Takebe. 1982. J. Gen. Virol. 63: 457-467.

10. Mol, J., R. van Blokland and J. Looter. 1991. Trends Biotechnol. 9: 182-183.

11. Paszty, C. J. R. and P. F. Lurquin. 1990. Plant Sci. 72: 69-79.

12. Peach, C. and J. Velten. 1991. Plant Mol. Biol. 17: 49-60.

13. Proudfoot, N. J. 1986. Nature 322: 562-565.

14. Reichel, C., J. Mathur, P. Eckes, K. Langenkemper, C. Koncz, J. Schell, B. Reiss and C. Maas. 1996. Proc. Natl. Acad. Sci. USA 93: 5888-5893.

15. Sambrook, J., E. F. Fritsch and T. Maniatis. 1989. Molecular cloning: A laboratory manual (2^{nd} Edition). Cold Spring Harbor Laboratory Press.

16. Thompson, A. J. and S. C. Myatt. 1997. Plant Mol. Biol. 34: 687-692.

17. Verdaguer, B., K. Alexandre, R. N. Beachy and C. Fauquet. 1996. Plant Mol. Biol. 31: 1129-1139.

18. Gatz, C. 1997. Annu. Rev. Plant Physiol. Plant Mol. Biol., 48:89-108.

19. Gatz, C. & Lenk, I. 1998. Trends Plant Sci., 3:352-358.

20. Zuo, J. & Chua, N-H. 2000. Current Opinion Biotech., 11:146-151.

21. Gatz, C. et al. 1992. Plant J., 2:397-404.

22. Weinmann, P. et al. 1994. Plant J., 5:559-569.

23. Caddick, M. X. et al. 1998. Nat. Biotechnol., 16:177-180.

24. Martinez, A. et al. 1999. Plant J., 19:97-106.

25. Bruce, W. et al. 2000. Plant Cell, 12:65-80.

26. Bohner, S. et al. 1999. Plant J., 19:87-95.

27. Mangelsdorf, D. J. et al. 1995. Cell, 83:835-839.

I claim:

1. A method to reduce transcriptional interference between two or more tandemly arranged gene expression cassettes in a host cell comprising introducing into the host cell a polynucleotide comprising a) a first gene expression cassette encoding a first polypeptide, b) a spacer polynucleotide, and c) a second gene expression cassette encoding a second polypeptide, whereby the first gene expression cassette and the second gene expression cassette are positioned in a tandem orientation and the spacer polynucleotide of b) is positioned between the first expression cassette and the second expression cassette; and culturing the host cell under conditions, whereby transcriptional interference between the first gene expression cassette and the second gene expression cassette is reduced and the first polypeptide and the second polypeptide are expressed.

2. The method according to claim 1, wherein the positioning of the first gene expression cassette and the second gene expression cassette is in an orientation selected from the group consisting of head (5′)-to-tail (3′) orientation, head (5′)-to-head (5′) orientation and tail (3′)-to-tail (3′) orientation.

3. The method according to claim 1, wherein the spacer polynucleotide of b) is selected from the group consisting of:
(i) a polynucleotide comprising a nucleic acid sequence of SEQ ID NO: 1;
(ii) a polynucleotide comprising a nucleic acid sequence of SEQ ID NO: 2;
(iii) a polynucleotide comprising a nucleic acid sequence of SEQ ID NO: 3; and
(iv) a polynucleotide comprising a nucleic acid sequence of SEQ ID NO: 4.

4. The method according to claim 1, wherein the spacer polynucleotide of b) comprises at least a 40% adenine and thiamine nucleotide content.

5. The method according to claim 1, wherein the spacer polynucleotide of b) comprises at least a 46% adenine and thiamine nucleotide content.

6. The method according to claim 1, wherein the spacer polynucleotide of b) comprises at least a 48% adenine and thiamine nucleotide content.

7. The method according to claim 1, wherein the spacer polynucleotide of b) comprises at least a 63% adenine and thiamine nucleotide content.

8. The method according to claim 1, wherein the host cell is selected from the group consisting of a bacterial, fungal, yeast, plant, animal and mammalian cell.

9. The method according to claim 8, wherein the plant cell is selected from the group consisting of an apple, Arabidopsis, bajra, banana, barley, bean, beet, blackgram, chickpea, chili, cucumber, eggplant, favabean, maize, melon, millet, mungbean, oat, okra, Panicum, papaya, peanut, pea, pepper, pigeonpea, pineapple, Phaseolus, potato, pumpkin, rice, sorghum, soybean, squash, sugarcane, sugarbeet, sunflower, sweet potato, tea, tomato, tobacco, watermelon, and wheat cell.

10. The method according to claim 1, wherein at least one of the gene expression cassettes comprises a polynucleotide encoding a polypeptide selected from the group consisting of an antigen, an alpha-amylase, a phytase, a glucane, a xylase, an insect resistance, a nematode resistance, a fungus resistance, a bacterium resistance, a virus resistance, an abiotic stress resistance, a nutraceutical, a pharmaceutical, an amino acid content modifying, a herbicide resistance, a cold tolerance, a drought tolerance, a heat tolerance, and an antioxidant polypeptide.

11. A host cell produced by a method comprising:
(a) introducing into the host cell a polynucleotide comprising (i) a first gene expression cassette encoding a first polypeptide, (ii) a spacer polynucleotide, and (iii) a second gene expression cassette encoding a second polypeptide, whereby the first gene expression cassette and the second gene expression cassette are positioned in a tandem orientation and the spacer polynucleotide of (ii) is positioned between the first expression cassette and the second expression cassette; and
(b) culturing the host cell under conditions, whereby transcriptional interference between the first gene expression cassette and the second gene expression cassette is reduced and the first polypeptide and the second polypeptide are expressed.

12. A non-human organism comprising the host cell of claim 11.

13. The non-human organism according to claim 12, wherein the non-human organism is selected from the group consisting of a bacterium, a fungus, a yeast, a plant, an animal, and a mammal.

14. The non-human organism according to claim 12, wherein the non-human organism is selected from the group consisting of an apple, Arabidopsis, bajra, banana, barley, bean, beet, blackgram, chickpea, chili, cucumber, eggplant, favabean, maize, melon, millet, mungbean, oat, okra, Panicum, papaya, peanut, pea, pepper, pigeonpea, pineapple, Phaseolus, potato, pumpkin, rice, sorghum, soybean, squash, sugarcane, sugarbeet, sunflower, sweet potato, tea, tomato, tobacco, watermelon, and wheat plant.


SEQUENCE LISTING (4 sequences)

SEQ ID NO: 1 - DNA, 154 bp, Artificial Sequence
  transcription blocker sequence from pSEAP2 vector
aataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 2 - DNA, 702 bp, Bacteriophage lambda
ggtaaccgtcgtattcccggcgcgtttattcagcaactgaaaaatggccggtggcatgtcatgcagcgtgtggctgggaaaaaccgttaccccattgatgtggtgaaaatcccgatggcggtgccgctgaccacggcgtttaaacaaaatattgagcggatacggcgtgaacgtcttccgaaagagctgggctatgcgctgcagcatcaactgaggatggtaataaagcgatgaaacatactgaactccgtgcagccgtactggatgcactggagaagcatgacaccggggcgacgttttttgatggtcgccccgctgtttttgatgaggcggattttccggcagttgccgtttatctcaccggcgctgaatacacgggcgaagagctggacagcgatacctggcaggcggagctgcatatcgaagttttcctgcctgctcaggtgccggattcagagctggatgcgtggatggagtcccggatttatccggtgatgagcgatatcccggcactgtcagatttgatcaccagtatggtggccagcggctatgactaccggcgcgacgatgatgcgggcttgtggagttcagccgatctgacttatgtcattacctatgaaatgtgaggacgctatgcctgtaccaaatcctacaatgccggtgaaaggtgccgggaccaccctgtgggtttataaggggagc

SEQ ID NO: 3 - DNA, 1519 bp, Bacteriophage lambda
gccttcacgcttcatacgcgggtcatagttggcaaagtaccaggcattttttcgcgtcacccacatgctgtactgcacctgggccatgtaagctgactttatggcctcgaaaccaccgagccggaacttcatgaaatcccgggaggtaaacgggcatttcagttcaaggccgttgccgtcactgcataaaccatcgggagagcaggcggtacgcatactttcgtcgcgatagatgatcggggattcagtaacattcacgccggaagtgaattcaaacagggttctggcgtcgttctcgtactgttttccccaggccagtgctttagcgttaacttccggagccacaccggtgcaaacctcagcaagcagggtgtggaagtaggacattttcatgtcaggccacttctttccggagcggggttttgctatcacgttgtgaacttctgaagcggtgatgacgccgagccgtaatttgtgccacgcatcatccccctgttcgacagctctcacatcgatcccggtacgctgcaggataatgtccggtgtcatgctgccaccttctgctctgcggctttctgtttcaggaatccaagagcttttactgcttcggcctgtgtcagttctgacgatgcacgaatgtcgcggcgaaatatctgggaacagagcggcaataagtcgtcatcccatgttttatccagggcgatcagcagagtgttaatctcctgcatggtttcatcgttaaccggagtgatgtcgcgttccggctgacgttctgcagtgtatgcagtattttcgacaatgcgctcggcttcatccttgtcatagataccagcaaatccgaaggccagacgggcacactgaatcatggctttatgacgtaacatccgtttgggatgcgactgccacggccccgtgatttctctgccttcgcgagttttgaatggttcgcggcggcattcatccatccattcggtaacgcagatcggatgattacggtccttgcggtaaatccggcatgtacaggattcattgtcctgctcaaagtccatgccatcaaactgctggttttcattgatgatgcgggaccagccatcaacgcccaccaccggaacgatgccattctgcttatcaggaaaggcgtaaatttctttcgtccacggattaaggccgtactggttggcaacgatcagtaatgcgatgaactgcgcatcgctggcatcacctttaaatgccgtctggcgaagagtggtgatcagttcctgtgggtcgacagaatccatgccgacacgttcagccagcttcccagccagcgttgcgagtgcagtactcattcgttttatacctctgaatcaatatcaacctggtggtgagcaatggtttcaaccatgtaccggatgtgttctgccatgcgctcctgaaactcaacatcgtcatcaaacgcacgggtaatggattttttgctggccccgtggcgttgcaaatgatcgatgcatagcgattcaaacaggtgctggggcag

SEQ ID NO: 4 - DNA, 2322 bp, Bacteriophage lambda
aagcttttctaatttaacctttgtcaggttaccaactactaaggttgtaggctcaagagggtgtgtcctgtcgtaggtaaataactgacctgtcgagcttaatattctatattgttgttctttctgcaaaaaagtggggaagtgagtaatgaaattatttctaacatttatctgcatcataccttccgagcatttattaagcatttcgctataagttctcgctggaagaggtagttttttcattgtactttaccttcatctctgttcattatcatcgcttttaaaacggttcgaccttctaatcctatctgaccattataattttttagaatggtttcataagaaagctctgaatcaacggactgcgataataagtggtggtatccagaatttgtcacttcaagtaaaaacacctcacgagttaaaacacctaagttctcaccgaatgtctcaatatccggacggataatatttattgcttctcttgaccgtaggactttccacatgcaggattttggaacctcttgcagtactactggggaatgagttgcaattattgctacaccattgcgtgcatcgagtaagtcgcttaatgttcgtaaaaaagcagagagcaaaggtggatgcagatgaacctctggttcatcgaataaaactaatgacttttcgccaacgacatctactaatcttgtgatagtaaataaaacaattgcatgtccagagctcattcgaagcagatatttctggatattgtcataaaacaatttagtgaatttatcatcgtccacttgaatctgtggttcattacgtcttaactcttcatatttagaaatgaggctgatgagttccatatttgaaaagttttcatcactacttagttttttgatagcttcaagccagagttgtctttttctatctactctcatacaaccaataaatgctgaaatgaattctaagcggagatcgcctagtgattttaaactattgctggcagcattcttgagtccaatataaaagtattgtgtaccttttgctgggtcaggttgttctttaggaggagtaaaaggatcaaatgcactaaacgaaactgaaacaagcgatcgaaaatatccctttgggattcttgactcgataagtctattattttcagagaaaaaatattcattgttttctgggttggtgattgcaccaatcattccattcaaaattgttgttttaccacacccattccgcccgataaaagcatgaatgttcgtgctgggcatagaattaaccgtcacctcaaaaggtatagttaaatcactgaatccgggagcactttttctattaaatgaaaagtggaaatctgacaattctggcaaaccatttaacacacgtgcgaactgtccatgaatttctgaaagagttacccctctaagtaatgaggtgttaaggacgctttcattttcaatgtcggctaatcgatttggccatactactaaatcctgaatagctttaagaaggttatgtttaaaaccatcgcttaatttgctgagattaacatagtagtcaatgctttcacctaaggaaaaaaacatttcagggagttgactgaattttttatctattaatgaataagtgcttacttcttctttttgacctacaaaaccaattttaacatttccgatatcgcatttttcaccatgctcatcaaagacagtaagataaaacattgtaacaaaggaatagtcattccaaccatctgctcgtaggaatgccttatttttttctactgcaggaatatacccgcctctttcaataacactaaactccaacatatagtaacccttaattttattaaaataaccgcaatttatttggcggcaacacaggatctctcttttaagttactctctattacatacgttttccatctaaaaattagtagtattgaacttaacggggcatcgtattgtagttttccatatttagctttctgcttccttttggataacccactgttattcatgttgcatggtgcactgtttataccaacgatatagtctattaatgcatatatagtatcgccgaacgattagctcttcaggcttctgaagaagcgtttcaagtactaataagccgatagatagccacggacttcgtagccatttttcataagtgttaacttccgctcctcgctcataacagacattcactacagttatggcggaaaggtatgcatgctgggtgtggggaagtcgtgaaagaaaagaagtcagctgcgtcgtttgacatcactgctatcttcttactggttatgcaggtcgtagtgggtggcacaca